(19) United States
(12) Patent Application Publication
(10) Pub. No.: US 2005/0074777 A1
(43) Pub. Date: Apr. 7, 2005

(54) Novel cell-surface estrogen receptor and related compositions and methods

(76) Inventor: C. Dominique Toran-Allerand, New York, NY (US)

Correspondence Address: John P. White;Cooper & Dunham LLP, 1185 Avenue of the Americas, New York, NY, 10036, US

(21) Appl. No.: 10/665,847

(22) Filed: Sep. 19, 2003

Related U.S. Application Data
(60) Provisional application No. 60/413,044, filed on Sep. 24, 2002.

(51) Int. Cl. C12Q 1/68; G01N 33/53; G01N 33/567; C07H 21/04; C07K 14/72

(52) U.S. Cl. 435/6; 435/69.1; 435/320.1; 435/325; 435/7.2; 530/350; 536/23.5

(57) ABSTRACT

This invention provides an isolated mammalian cell-surface estrogen receptor characterized by (a) a non-stereospecific binding affinity for 17α-estradiol and 17β-estradiol; (b) at least one epitope in common with the ligand-binding domain of ER-α; and (c) increased presence at caveolar or caveolar-like microdomains of cells on which the receptor is present. This invention further provides related methods for assaying compounds, activating the MAP kinase pathway of a cell, and delaying the onset of or treating disorders. Finally, this invention provides related articles and compositions of matter.

This invention was made with funding from the United States National Institute of Aging (NIA), Grant No. 1ROIAG-15092, and National Institute of Mental Health (NIMH), Grants No. 1ROIMH49682 and 5KO5 MH-00192. Accordingly, the United States Government has certain rights in this invention.

Throughout this application, various publications are referenced. Full bibliographic citations for these publications are found at the end of the specification immediately preceding the claims. The disclosures of these publications in their entirety are hereby incorporated by reference into this application.

### BACKGROUND OF THE INVENTION

Estrogen and Estrogen Receptors Generally

Tissue targets of estrogen include the reproductive tract, breast, cardio and cerebrovascular systems and central nervous system (CNS). Estrogen is an important neural growth and trophic factor with influences on neuronal development, survival, and plasticity throughout life (Toran-Allerand, 1996).

There are now at least two mammalian estrogen receptor (ER) genes encoding, respectively, the “classical” receptor ER-α (mouse ER-α, ˜67 kDa), which mediates most of estrogen's known transcriptional actions in the brain (White, 1987), and the more recently cloned ER-β (mouse ER-β, ˜60 kDa), whose neural role remains largely uncharacterized but may be modulatory (Kuiper, 1996; and Tremblay, 1997). A third, more distantly related member of the ER family, ER-γ, was cloned in teleosts (Hawkins, 2000). ER-α and ER-β appear to be complementary but not redundant. Under steady-state conditions, ER-α and ER-β are predominantly intranuclear and differ to varying degrees with respect to the homology of their functional domains, binding affinities and ligand specificities (Kuiper, 1997).

The spatio-temporal expression and distribution of ER-α and ER-β differ with developmental stage. For example, neocortical ER-β is present throughout life, whereas neocortical ER-α expression is developmentally regulated and normally expressed at very high levels only during the period of neocortical differentiation, suggesting a more restricted developmental role (Gerlach, 1983; Shughrue, 1990; and Shughrue, 1997).

The theoretical existence of membrane ERs has been suggested in the literature for the past twenty-five years (Anuradha, 1994; Pietras, 1977; and Watson, 1999). However, to date, no conclusive evidence has demonstrated whether these theoretical membrane ERs exist as a small subpopulation of both ER-α (Watson, 1999) or ER-β, or in fact represent novel members of the ER family (Das, 1997; and Gu, 1999). Singh et al. and Toran-Allerand have suggested that an estrogen receptor subtype, designated “ER-X”, would be expected to exist in neocortical cells, but have provided no characterization of this suggested entity (Singh, 1999; Singh, 2000; and Toran-Allerand, 2000).

Estrogen Signaling

The traditional view of estrogen action is that the intranuclear ERs act as ligand-inducible, transcriptional enhancers which, on binding to cognate response elements in DNA, regulate a wide variety of transcription factors and genes by either enhancing or suppressing their function (Beato, 2000; and Landers, 1992). Some responses to estradiol cannot be attributed to ER-α or ER-β such as estrogen's ability to regulate non-ERE-containing genes and the very rapid (seconds to minutes) effects of estrogen (Chiaia, 1983; Garcia-Segura, 1987; Kelly, 1978; Migliaccio, 1993; Singh, 1999; Singh, 2000; and Sukovich, 1994). While such rapid responses appear inconsistent with direct transcriptional modulation via intranuclear receptors, they could be explained by the presence of plasma membrane-associated ERs that may be coupled to signal transduction pathways, typically associated with rapid activation by growth factors.

In addition to its well described transcriptional actions, estrogen has been shown to activate classical second messengers, including cAMP (Aronica, 1994), inositol phosphate and calcium (Guo, 2002; and Marino, 2001). It has also been shown that estrogen elicits rapid activation of signaling pathways such as the MAPK cascade (Singh, 1999; Singer, 1999; and Singh, 2000) and the phosphoinositide-3 (PI-3) kinase/Akt (protein kinase B) pathway (Singh, 2001). These signaling pathways are typically thought to be associated with membrane growth factor receptor tyrosine kinases or coupled to heptahelical membrane receptors and heterotrimeric G-proteins (Gutkind, 2000). Although the molecular events which follow estrogen binding to its receptors in the brain are poorly understood, some of estrogen's actions in the developing brain rely on signal transduction mechanisms that originate at the plasma-membrane and are broadly similar to those that underlie the actions of growth factors such as the neurotrophins (Aronica, 1994; Singh, 1999; and Singh, 2000).

Neurotrophins and numerous other growth factors have been shown to be important to neuronal differentiation and survival. Neurotrophin activation of the MAPK cascade is mediated by cognate transmembrane receptors associated with caveolar-like microdomains (CLMs) of neuronal plasma-membranes (Huang, 1999). Caveolar-like microdomains (CLMs) are the neuron-specific homologues of caveolae which are microdomains associated with the plasma-membrane of most cell types (Anderson, 1998; Okamoto, 1998; and Schlegel, 1998). However, unlike caveolae proper, CLMs express the integral membrane protein flotillin (Bickel, 1997) rather than the caveolar protein, caveolin. CLMs, like caveolae, are highly enriched in cholesterol, glycosphingolipids, sphingomyelin and lipid-anchored membrane proteins and have been implicated in signal transduction and lipid/protein trafficking. Numerous molecules involved in growth factor- and neurotransmitter-induced cell signaling, such as receptor tyrosine kinases, the src family, members of the MAPK cascade, and G-proteins/G-protein-coupled receptors, among many others (Schlegel, 1998), have been identified in CLMs and caveolae, suggesting that these may serve as functional signaling modules to compartmentalize, modulate and integrate signaling events at the cell surface.

Like the neurotrophins, estrogen is an important neural trophic factor throughout life, with influences on neuronal differentiation (Toran-Allerand, 1976; and Toran-Allerand, 1980), survival (Garcia-Segura, 2001; and Green, 2000), and plasticity (Matsumoto, 1981). 17β-estradiol activates many signaling kinases including protein kinase C (PKC), c-src (Nethrapalli, 2001) and members of the MAPK cascade (Singh, 1999; Singh, 2000; and Watters, 1997). Rapid and sustained activation of cytoplasmic ERK1/2 is followed by nuclear translocation of phosphorylated ERK (Sétáló, 2001).

Although both estrogen and the neurotrophin BDNF elicit rapid and sustained activation of the MAPK cascade (Singh, 1999), accompanied by nuclear translocation of the phosphorylated ERKs (Sétáló, 2002), the pathways leading to ERK1/2 activation are not identical. Some components of the cascade are shared in common while others differ. The significance of these differences is unknown. 17β-estradiol activation of ERK1/2 is initiated via PLCγ and PI-3 kinase, PKC and c-src (Nethrapalli, 2001). However, unlike BDNF, such activation is not dependent on protein kinase A (PKA) or Ca⁺⁺. Estrogen-induced PKC activation is followed sequentially by rapid activation of Ras, B-Raf (but not Raf-1 (c-Raf) or Rap1) and MEK2 (but not MEK1). Both estrogen and BDNF then activate MAP kinase family members, including ERK1 and ERK2, which are involved in neuronal differentiation (Marshall, 1995; and Traverse, 1992), and ERK5, which is involved with neuronal survival (Watson, 2001). While BDNF activates p38 and c-jun N-terminal kinase (JNK), estrogen does not. Although the significance of preferential activation is unknown, cross-coupling or convergence of the estrogen and neurotrophin signaling pathways may not simply represent an overlap of signaling sequelae but, rather, depicts a unique pathway or pathways for estrogen's actions in the brain that could be instrumental in the developmental and neuroprotective actions of estrogen.

Effects of Estrogen on the Central Nervous System (CNS)

Estrogen has been shown to play an integral role in brain development, neural plasticity, neuroprotection and neural repair. The influence of estrogen on the brain has considerable relevance for the mechanisms underlying (i) estrogen actions on higher order cognitive processes; (ii) the genesis of the sexually dimorphic childhood disorders of cognition (e.g., learning disabilities, infantile autism), delayed speech acquisition, and attention deficit disorder (Geschwind, 1982; Tallal, 1991a; and Tallal, 1991b); (iii) neurodevelopmental disorders with cognitive deficits, e.g., schizophrenia (Arnold, 1996; and Strauss, 1992), and Turner's (XO) syndrome (Jones, 1995); and (iv) the dementias associated with Down's syndrome (trisomy 21) (Pennington, 1985), and Alzheimer's and Parkinson's diseases (Schupf, 2002; and Tang, 1996). These conditions are of considerable clinical, economic and educational importance.

While the basis for the striking male predominance in the incidence of the sexually dimorphic disorders is unclear, differences in cortical maturation rates, arising from sex differences in androgen levels, may be causative (Tallal, 1991a; and Tallal, 1991b). This view is supported by studies in developing primates which have shown potentiation of behavioral deficits in response to lesions of the orbital prefrontal cortex, following early androgen exposure (males >> females) (Tsuchiya, 2002). By accelerating maturation of this brain region, testosterone, acting directly or following aromatization to estradiol, may be responsible for the observed sex differences in the recovery (plasticity) from such lesions (Goldman, 1974). Transient localization of the highest levels of cortical aromatase activity and of estrogen binding to the association cortex, which includes the orbital prefrontal cortex, provides a basis for understanding how these estrogenic androgens might influence the development of the primate neocortex, particularly areas of the association cortex whose interconnections form a neural system which may be important for cognitive functions and may be involved in the cognitive deficits associated with schizophrenia (Clark, 1989; and MacLusky, 1986).

Estrogen in Uterine and Pulmonary Development

Turner's syndrome (XO) is a genetic disorder effecting both neurodevelopmental and sexual development. In Turner's syndrome, the fetus is supplied in utero with estrogen from the mother. Shortly after birth, however, the ovaries become fibrotic and no estrogen is produced. As a result of the absence of estrogen, secondary sex characteristics do not develop in girls with Turner's syndrome. The current treatment for Turner's syndrome is administration of Premarin (pregnant mare urine, Wyeth) at the age when the onset of puberty should normally occur. However, with this treatment only 50% of the girls develop a normal uterus. In preliminary clinical trials using an estradiol patch (17β-estradiol), girls with Turner's syndrome had nearly normal uterine development. Large-scale testing of the use of estradiol to treat Turner's syndrome has not been conducted and therefore the non-specific effects of 17β-estradiol and potentially dangerous side effects (e.g., blood clots, and enhanced growth of pre-existing cancers) of this treatment have not been evaluated. Development of safer, more specific drugs which target estrogen receptors in the CNS and uterus are needed to improve treatment of Turner's syndrome.

Estrogen also effects pulmonary development. During pregnancy there is a hundred-fold increase in 17β-estradiol and progesterone plasma concentrations in both the mother and the fetus (Trotter, 2000). The placental supply of these hormones is disrupted at birth. Preterm infants are therefore deprived of this hormonal supply at an earlier developmental stage than full-term infants. Steroid hormones have been shown to promote lung development. Preterm infants are usually treated with glucocorticoids to aid in lung development, but this treatment has potential risk factors such as seizures and other steroid-related complications.

In recent clinical trials, replacement doses of progesterone and 17β-estradiol were administered to preterm infants and treatment resulted in a decrease in the incidence of lung disorders (Trotter, 2000). In utero administration of estrogen has been shown to stimulate both the formation and release of surfactant in rat fetal lungs (Thuresson-Klein, 1985). Drugs tailored to specifically target lung estrogen receptors may offer alternative and safe treatment options to stimulate lung development in preterm infants.

### SUMMARY OF THE INVENTION

This invention provides an isolated mammalian cell-surface estrogen receptor characterized by (a) a non-stereospecific binding affinity for 17α-estradiol and 17β-estradiol; (b) at least one epitope in common with the ligand-binding domain of ER-α; and (c) increased presence at caveolar or caveolar-like microdomains of cells on which the receptor is present.

This invention further provides a composition of matter comprising a lipid membrane, other than that of an intact cell, comprising instant the receptor operably situated therein.

This invention further provides a method for determining whether an agent specifically binds to the instant receptor which comprises (a) contacting the receptor with the agent under suitable conditions; (b) detecting the presence of any complex formed between the receptor and the agent; and (c) determining whether the complex detected in step (b) is the result of specific binding between the agent and receptor, thereby determining whether the agent specifically binds to the receptor.

This invention further provides a method for determining the affinity with which an agent binds to the instant receptor relative to that with which a known ligand binds the receptor, which comprises (a) concurrently contacting the receptor with both the agent and a ligand that binds the receptor with a known affinity under conditions which permit the formation of a complex between the receptor and the ligand; (b) determining the amount of complex formed between the agent and the receptor; and (c) comparing the amount of complex determined in step (b) with the amount of complex formed between the agent and the receptor in the absence of the ligand, wherein (i) a ratio of agent in the complex determined in step (c) to that determined in step (b) greater than 2 indicates that the agent binds to the receptor with less affinity than does the ligand, (ii) a ratio of less than 2 indicates that the agent binds to the receptor with greater affinity than does the ligand, and (iii) a ratio of 2 indicates that the agent and ligand bind to the receptor with the same affinity.

This invention further provides a method for determining whether an agent is an agonist of the instant receptor, which comprises (a) contacting the receptor with the agent under conditions which permit (i) the formation of a complex between the receptor and a known agonist of the receptor, and (ii) the generation of a detectable signal upon formation of a complex between the receptor and the known agonist; and (b) determining whether a detectable signal is generated in step (a), the generation of such signal indicating that the agent is an agonist of the receptor.

This invention further provides a method for determining whether an agent is an antagonist of the instant receptor, which comprises (a) contacting the receptor with the agent, in the presence of a known agonist, under conditions which permit (i) the formation of a complex between the receptor and the agonist, and (ii) the generation of a detectable signal upon formation of a complex between the receptor and the agonist; and (b) comparing the signal, if any, generated in step (a) with the signal generated in the absence of the agent, the generation of a signal in the agent's absence greater than that generated in the agent's presence indicating that the agent is an antagonist.

This invention further provides a method for activating the MAP kinase pathway of a cell having on its surface the instant receptor comprising contacting the cell with a concentration of 17α-estradiol of at least 0.1 pM and less than 100 pM under conditions permitting the 17α-estradiol to bind to the receptor, thereby activating the MAP kinase pathway in the cell.

This invention further provides a method for treating a subject afflicted with a neurodegenerative disorder, comprising administering to the subject an amount of 17α-estradiol sufficient to raise the subject's plasma 17α-estradiol concentration to at least 0.1 pM and less than 100 pM, thereby treating the subject.

This invention further provides a method for delaying the onset of a neurodegenerative disorder in a subject, comprising administering to the subject an amount of 17α-estradiol sufficient to raise the subject's plasma 17α-estradiol concentration to at least 0.1 pM and less than 100 pM, thereby delaying the onset of the neurodegenerative disorder in the subject.

This invention further provides a method for treating a subject afflicted with a neurodevelopmental disorder, comprising administering to the subject an amount of 17α-estradiol sufficient to raise the subject's plasma 17α-estradiol concentration to at least 0.1 pM and less than 100 pM, thereby treating the subject.

This invention further provides a method for treating a subject afflicted with a sexually dimorphic childhood disorder of cognition, comprising administering to the subject an amount of 17α-estradiol sufficient to raise the subject's plasma 17α-estradiol concentration to at least 0.1 pM and less than 100 pM, thereby treating the subject.

This invention further provides a method for treating a subject afflicted with a uterine disorder, comprising administering to the subject an amount of 17α-estradiol sufficient to raise the subject's plasma 17α-estradiol concentration to at least 0.1 pM and less than 100 pM, thereby treating the uterine disorder in the subject.

This invention further provides a method for treating a subject afflicted with a pulmonary disorder, comprising administering to the subject an amount of 17α-estradiol sufficient to raise the subject's plasma 17α-estradiol concentration to at least 0.1 pM and less than 100 pM, thereby treating the subject.

This invention further provides a composition comprising (a) a pharmaceutically acceptable carrier and (b) a dose of 17α-estradiol which, when administered to a subject, is sufficient to raise the subject's plasma 17α-estradiol concentration to at least 0.1 pM and less than 100 pM.

Finally, this invention provides an article of manufacture comprising (a) a packaging material having therein an amount of 17α-estradiol sufficient, upon administration to a subject, to raise the subject's plasma 17α-estradiol concentration to at least 0.1 pM and less than 100 pM, and (b) a label indicating a use of the 17α-estradiol for treating a disorder selected from the group consisting of a neurodegenerative disorder, a neurodevelopmental disorder, a sexually dimorphic childhood disorder of cognition, a uterine disorder, and a pulmonary disorder.

### BRIEF DESCRIPTION OF THE FIGURES

FIG. 1

ER-X is neither ER-α nor ER-β. (a) Western immunoblots of P7 wild-type and ERKO neocortex and adult wild-type mouse ovary, using antibodies to the LBDs of ER-α (Santa Cruz; MC-20; ovary and neocortex) and ER-β (Zymed; ovary). The apparent molecular weight (MW) of mouse ER-X (˜62-63 kDa) is clearly different from the MW of the mouse ER-α (67 kDa) and mouse ER-β (60 kDa) ovarian controls. (b) While P7 wild-type neocortex contained both the 67 kDa ER-α and the ˜62-63 kDa ER-X bands, P7 ERKO tissues expressed only the ˜62-63 kDa ER-X band. P7 wild-type and ERKO neocortical CLM preparations were greatly enriched with the 62-63 kDa protein. A striking reversal of the ER-α/ER-X ratio was seen in wild-type CLM preparations, in which the 62-63 kDa form was highly enriched, while authentic 67 kDa ER-α was considerably diminished. (c) Absence of ER-β from the plasma membrane, CLM and non-CLM regions. Note the total absence of ER-β from the ERKO plasma membrane and the CLM and non-CLM fractions. Note also the nuclear concentration of the 60 and 64 kDa isoforms of ER-β. PM, plasma-membrane; non-CLM, non-caveolar-like membrane; CLM, caveolar-like membrane.

FIG. 2

Characterization and purity of the CLM preparations. (a) Western immunoblots of CLMs show enrichment in flotillin, the neuron-specific, integral CLM protein. The purity of CLM preparations was verified (b) by the presence of caveolar-enriched resident proteins such as PKC-α, and (c) by the absence of the cytosolic protein paxillin, a cytoskeletal component associated with non-CLM regions.

FIG. 3

ER-X is exquisitely sensitive to picoMolar (pM) concentrations of 17α-estradiol and 17β-estradiol. Upper blots: Western immunoblot of ERK1/2 phosphorylation elicited in wild-type neocortical explants by (a) 17β-estradiol and (b) 17α-estradiol. Lower blots: Re-probing with antibodies to total non-phosphorylated ERK1/2 to verify equal loading of ERK1/2 protein across lanes. (pERK=phosphoERK). Densitometry confirmed equal loading. Note that significantly higher levels of 17β-estradiol were required for ERK activation, perhaps reflecting the need in wild-type cultures to overcome the inhibitory effect of ER-α on ERK phosphorylation which, unlike 17α-estradiol, 17β-estradiol activates as well.

FIG. 4

Estrogen-induced activation of ERK1/2 in CLMs and post-nuclear supernatant (PNS). Upper blots: (a) exposure of highly purified, P7 ERKO neocortical CLMs to 17α-estradiol (0.1 nM) and 17β-estradiol (10 nM) for 30 minutes elicited MEK-dependent (U0126) phosphorylation of ERK1 and ERK2 (pERK=phosphoERK). Non-CLM regions were unresponsive. Densitometry confirmed equal loading of protein. (b) Exposure of P7 wild-type neocortical PNS to 17α-estradiol (0.1 nM) and 17β-estradiol (10 nM) for 10 minutes, 37° C. elicited MEK-dependent (U0126) phosphorylation of ERK1 and ERK2. Note that not only did the ER-α-selective ligand PPT reduce ERK phosphorylation levels below baseline (0) very significantly, but the level of ERK1/2 phosphorylation, elicited by 17β-estradiol, was also significantly lower than following exposure to 17α-estradiol. This difference may be attributed to the fact that P7 wild-type neocortex is also enriched in ER-α which, since it is activated by 17β-estradiol (but not 17α-estradiol), exerts its inhibitory effect on ERK1/2, as was also seen following exposure to propylpyrazole triol (PPT). Lower blots: Re-probing with antibodies to non-phosphorylated ERK1/2 to verify equal loading of ERK protein across lanes. (pERK=phosphoERK). Densitometry confirmed equal loading. (c) Densitometric analysis of ERK activation in wild-type PNS shown in (b). These findings confirm that ER-α is a strong inhibitor of ERK activation, a measure of which is shown by the ability of PPT to effectively prevent ERK activation even in the face of the strong activation of ERK elicited by the PPT vehicle ethanol.

FIG. 5

Disruption of cholesterol in CLMs impairs ERK activation. Upper blots: Selective disruption of membrane cholesterol by Nystatin in 9 day-old wild-type neocortical explants decreased the ability of estradiol and the BDNF control to elicit ERK phosphorylation. Lower blots: Re-probing with antibodies to non-phosphorylated ERK1/2 to verify equal loading of ERK protein across lanes. (pERK=phosphoERK). Densitometry confirmed equal loading.

FIG. 6

ER-X has homology with the LBD of ER-α. Whole-mount of a P2 ERKO neocortical explant, 17 days in vitro. The culture was stained for ER-α mRNA by in situ hybridization with a 48 base oligonucleotide probe to an alpha-specific region of the ER-α LBD (BER2; Miranda, 1992) and shows the ER-α-like mRNA hybridization signal in neocortical neurons.

FIG. 7

Direct evidence in ERKO that ER-X is a neuronal plasma-membrane-associated receptor with some homology to the ER-α LBD. (a) Using antibodies highly specific for an alpha-specific region of the LBD of ER-α (C1355), large numbers of immature immunoreactive neocortical ERKO neurons with unstained nuclei are seen. (b) The immunoreactivity is clearly localized to the cell membrane and cytoplasm, but not in the nucleus. (d and e) Antibodies, raised against the full-length ER-α molecule, said to recognize epitopes in the 5′, N-terminal region (6F11), but also cross-reacts significantly with ER-β, show widespread nuclear labeling with no cytoplasmic or membrane labeling seen. The nuclear labeling observed most likely reflects intranuclear ER-β which is normally expressed in both wild-type and ERKO neocortical neurons. (c) CLM association of ER-X in ERKO neocortical neurons was further documented at the ultrastructural level by demonstrating immunoreactive flotillin (gold particles), co-localized with immunoreactivity for the ER-α LBD (horseradish peroxidase) on a mushroom-like neocortical dendritic spine. Scale bars 10 μm.

FIG. 8

Binding of ³H estradiol to Percoll®-purified plasma-membranes from P7 ERKO and wild-type mouse neocortex. (a) Identical amounts of membrane protein (50 μg/tube) were incubated with varying concentrations of ³H estradiol (0.3-8 nM) for 18 hours at 4° C. The reaction was terminated by addition of hydroxylapatite (HAP). The membranes and HAP were sedimented by centrifugation in a microfuge, and the pellet washed 4 times to remove free steroid. Radioactivity in the pellets was extracted with ethanol and counted. Non-saturable binding, assessed in the presence of 1 μM unlabelled DES, was subtracted from the total counts and the saturable binding plotted as the ratio of bound/unbound ligand versus the concentration of bound ³H estradiol. Similar concentrations of high affinity binding (equilibrium dissociation constant, Kd, ˜1.6 nM) were observed in wild type and ERKO membranes. (b) Specificity of the binding site in Percoll®-purified membranes from P7 ERKO mouse neocortex. Aliquots of plasma-membrane were incubated with 2 nM ³H estradiol for 18 hours at 4° C. in the presence and absence of different concentrations (50 nM and 1 μM) of 17α-estradiol, 17β-estradiol or progesterone. Bound ³H estradiol was separated by sedimentation with HAP and counted at an efficiency of 50%. Data represent the number of bound counts (after subtraction of HAP-only blank control tubes, containing no membrane protein) expressed as the means +/−S.D. of triplicate determinations. The horizontal dashed line indicates the level of non-specific binding observed in the presence of 1 μM DES.

FIG. 9

ER-X is developmentally regulated. ER-X expression is developmentally regulated and is maximally expressed around P7-10 in (a) the neocortex and (b) the uterus. During the first postnatal month, wild-type and ERKO neocortical ER-X levels decline dramatically and become barely visible in the adult.

FIG. 10

ER-X is up-regulated following ischemic brain injury in the adult. Comparison of ER-α and ER-X expression in the infarcted and non-infarcted adult neocortex. Following a large ischemic infarct in the neocortex produced by middle cerebral artery occlusion, there was not only up-regulation of ER-1 expression in the penumbra of the ligated, ischemic side but also up-regulation of ER-X therein as well, suggesting re-expression of a developmental mechanism normally latent in the adult. Note the lack of significant ER-X expression on the non-infarcted side. (MCF-7 mammary tumour cells and adult uterus=ER-α controls; P7 neocortex=ER-X control).

### DETAILED DESCRIPTION OF THE INVENTION

Definitions

In this invention, “administering” can be effected or performed using any of the various methods and delivery systems known to those skilled in the art. The administering can be performed, for example, intravenously, orally, nasally, via implant, transmucosally, transdermally, intramuscularly, and subcutaneously. The following delivery systems, which employ a number of routinely used pharmaceutically acceptable carriers, are only representative of the many embodiments envisioned for administering the instant compositions.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

As used herein, the term “agent” shall include, without limitation, a nucleic acid, a steroid, a lipid, a carbohydrate moiety, a protein, a polypeptide, and a small molecule.

As used herein, the term “lipid membrane” includes, without limitation, a liposome, a lipid membrane fragment, and a plasma membrane of a cell which does not normally express the receptor.

As used herein, the term “operably situated” shall mean situated in a manner preserving the native function of that which is situated. For example, a receptor which is membrane-bound in nature, is operably situated in a lipid membrane if the receptor retains its ability to bind its natural ligand(s) and, as appropriate, undergo any conformational or other change undergone by the receptor in nature upon binding to its natural ligand(s). In one embodiment, a receptor operably situated in a plasma lipid membrane is in the same configuration as it is found in the membrane of a cell normally expressing such receptor.

As used herein, “non-steroespecific binding affinity” shall mean, in respect to a receptor, a binding affinity between the receptor and one stereoisomer of the receptor's ligand which is comparable to the binding affinity between the receptor and a different stereoisomer of the ligand. For example, a receptor which binds a first stereoisomer of its ligand with affinity X and binds a second stereoisomer of its ligand with an affinity of 0.5X-2X has a non-stereospecific binding affinity for the first and second stereoisomers of its ligand. However, a receptor which binds a first stereoisomer of its ligand with affinity X and binds a second stereoisomer of its ligand with an affinity of 100X does not have a non-stereospecific binding affinity for the first and second stereoisomers of its ligand.

“Pharmaceutically acceptable carriers” include, in addition to those listed above, and without limitation, 0.01-0.1M and preferably 0.05M phosphate buffer, phosphate-buffered saline, or 0.9% saline, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases and the like.

As used herein, the term “subject” shall mean any animal including, without limitation, a human, a mouse, a rat, a rabbit, a dog, a guinea pig, a ferret, a non-human primate, or any other mammal. In the preferred embodiment, the subject is human. The subject can be male or female.

Embodiments of the Invention

This invention provides an isolated mammalian cell-surface estrogen receptor characterized by (a) a non-stereospecific binding affinity for 17α-estradiol and 17β-estradiol; (b) at least one epitope in common with the ligand-binding domain of ER-α; and (c) increased presence at caveolar or caveolar-like microdomains of cells on which the receptor is present. In the preferred embodiment, the receptor is a human receptor.

This invention further provides a composition of matter comprising a lipid membrane, other than that of an intact cell, comprising the instant receptor operably situated therein. The instant receptor can be from any mammalian species and in the preferred embodiment, the receptor is a human receptor.

This invention further provides a method for determining whether an agent specifically binds to the instant receptor which comprises (a) contacting the receptor with the agent under suitable conditions; (b) detecting the presence of any complex formed between the receptor and the agent; and (c) determining whether the complex detected in step (b) is the result of specific binding between the agent and receptor, thereby determining whether the agent specifically binds to the receptor. In the preferred embodiment, the receptor is a human receptor. Also, the receptor is preferably operably situated within a lipid membrane.

This invention further provides a method for determining the affinity with which an agent binds to the instant receptor relative to that with which a known ligand binds the receptor, which comprises (a) concurrently contacting the receptor with both the agent and a ligand that binds the receptor with a known affinity under conditions which permit the formation of a complex between the receptor and the ligand; (b) determining the amount of complex formed between the agent and the receptor; and (c) comparing the amount of complex determined in step (b) with the amount of complex formed between the agent and the receptor in the absence of the ligand, wherein (i) a ratio of agent in the complex determined in step (c) to that determined in step (b) greater than 2 indicates that the agent binds to the receptor with less affinity than does the ligand, (ii) a ratio of less than 2 indicates that the agent binds to the receptor with greater affinity than does the ligand, and (iii) a ratio of 2 indicates that the agent and ligand bind to the receptor with the same affinity. In the preferred embodiment, the receptor is a human receptor. The known ligand is either 17β-estradiol or 17α-estradiol.

Methods for administering 17β-estradiol and 17α-estradiol are described in U.S. Pat. Nos. 5,843,934 and 5,554,601 which describe methods for neuroprotection and treatment of disease. Other ligands include, but are not limited to, bisphenol A and estriol.

The affinity with which an agent binds to a receptor can be measured using, for example, routine methods for determining dissociation constants and/or affinity constants.

This invention further provides a method for determining whether an agent is an agonist of the instant receptor which comprises (a) contacting the receptor with the agent under conditions which permit (i) the formation of a complex between the receptor and a known agonist of the receptor, and (ii) the generation of a detectable signal upon formation of a complex between the receptor and the known agonist; and (b) determining whether a detectable signal is generated in step (a), the generation of such signal indicating that the agent is an agonist of the receptor.

This invention further provides a method for determining whether an agent is an antagonist of the instant receptor, which comprises (a) contacting the receptor with the agent, in the presence of a known agonist, under conditions which permit (i) the formation of a complex between the receptor and the agonist, and (ii) the generation of a detectable signal upon formation of a complex between the receptor and the agonist; and (b) comparing the signal, if any, generated in step (a) with the signal generated in the absence of the agent, the generation of a signal in the agent's absence greater than that generated in the agent's presence indicating that the agent is an antagonist.

In one embodiment of these methods, the signal comprises an increase ERK1/2 phosphorylation. In another embodiment of these methods, the signal comprises an increase in MEK2 phosphorylation. Other signals include, but are not limited to, changes in CAMP and inositol phosphate levels, and activation of the MAPK cascade and the phospoinositide (PI-3) kinase/Akt (protein kinase B) pathway.

This invention further provides a method for activating the MAP kinase pathway of a cell having on its surface the instant receptor comprising contacting the cell with a concentration of 17α-estradiol of at least 0.1 pM and less than 100 pM under conditions permitting the 17α-estradiol to bind to the receptor, thereby activating the MAP kinase pathway in the cell. Cells include, for example, a neuronal cell, a uterine cell, a stem cell, and a pulmonary cell. In the preferred embodiment, the cell is a human cell.

This invention further provides a method for treating a subject afflicted with a neurodegenerative disorder, comprising administering to the subject an amount of 17α-estradiol sufficient to raise the subject's plasma 17α-estradiol concentration to at least 0.1 pM and less than 100 pM, thereby treating the subject. This invention further provides a method for delaying the onset of a neurodegenerative disorder in a subject, comprising administering to the subject an amount of 17α-estradiol sufficient to raise the subject's plasma 17α-estradiol concentration to at least 0.1 pM and less than 100 pM, thereby delaying the onset of the neurodegenerative disorder in the subject. Neurodegenerative disorders include, without limitation, stroke, Alzheimer's disease, Parkinson's disease, and Multiple Sclerosis. In the preferred embodiment, the subject is human.

This invention further provides a method for treating a subject afflicted with a neurodevelopmental disorder, comprising administering to the subject an amount of 17α-estradiol sufficient to raise the subject's plasma 17α-estradiol concentration to at least 0.1 pM and less than 100 pM, thereby treating the subject. Neurodevelopmental disorders include, without limitation, schizophrenia, Turner's syndrome, and Down's syndrome. In the preferred embodiment, the subject is human.

This invention further provides a method for treating a subject afflicted with a sexually dimorphic childhood disorder of cognition, comprising administering to the subject an amount of 17α-estradiol sufficient to raise the subject's plasma 17α-estradiol concentration to at least 0.1 pM and less than 100 pM, thereby treating the subject. In one embodiment, the sexually dimorphic childhood disorder of cognition is a learning disability. Sexually dimorphic childhood disorders of cognition include, without limitation, infantile autism, delayed speech acquisition, and attention deficit disorder. In the preferred embodiment, the subject is human.

This invention further provides a method for treating a subject afflicted with a uterine disorder, comprising administering to the subject an amount of 17α-estradiol sufficient to raise the subject's plasma 17α-estradiol concentration to at least 0.1 pM and less than 100 pM, thereby treating the uterine disorder in the subject. In one embodiment, the uterine disorder is Turner's syndrome. In the preferred embodiment, the subject is human.

This invention further provides a method for treating a subject afflicted with a pulmonary disorder, comprising administering to the subject an amount of 17α-estradiol sufficient to raise the subject's plasma 17α-estradiol concentration to at least 0.1 pM and less than 100 pM, thereby treating the subject. In one embodiment, the pulmonary disorder is immature lung development in a preterm infant. In the preferred embodiment, the subject is human.

In one embodiment of the instant methods, the amount of 17α-estradiol administered to the subject is an amount sufficient to raise the subject's plasma 17α-estradiol concentration to 0.1, 0.5, 1, 5, 10, 15, 20, 25, 30, 35, 40, 45, 50, 55, 60, 65, 70, 75, 80, 85, 90, or 95 pM. In another embodiment, the amount of 17α-estradiol administered to a subject is the amount sufficient to raise the subject's plasma 17α-estradiol concentration to between 0.1-20 pM. In another embodiment, the amount of 17α-estradiol administered to a subject is the amount sufficient to raise the subject's plasma 17α-estradiol concentration to between 1-10 pM. In a further embodiment, the amount of 17α-estradiol administered to a subject is the amount sufficient to raise the subject's plasma 17α-estradiol concentration to between 10-99 pM. In the instant methods, the amount of 17α-estradiol administered to a subject can also be the amount sufficient to raise the subject's plasma 17α-estradiol concentration to between 1 pM-1 nM, and to 1, 5, or 10 nM.

Determining an amount of 17α-estradiol sufficient to raise the subject's plasma 17α-estradiol concentration to a predetermined amount can be done based on animal data using routine computational methods such as radioimmunoassay methods.

This invention further provides a composition comprising (a) a pharmaceutically acceptable carrier and (b) a dose of 17α-estradiol which, when administered to a subject, is sufficient to raise the subject's plasma 17α-estradiol concentration to at least 0.1 pM and less than 100 pM.

Finally, this invention provides an article of manufacture comprising (a) a packaging material having therein an amount of 17α-estradiol sufficient, upon administration to a subject, to raise the subject's plasma 17α-estradiol concentration to at least 0.1 pM and less than 100 pM, and (b) a label indicating a use of the 17α-estradiol for treating a disorder selected from the group consisting of a neurodegenerative disorder, a neurodevelopmental disorder, a sexually dimorphic childhood disorder of cognition, a uterine disorder, and a pulmonary disorder.

This instant invention is illustrated in the Experimental Details section that follows. This section is set forth to aid in an understanding of the instant invention but is not intended to, and should not be construed to, limit in any way the invention as set forth in the claims which follow thereafter.

Experimental Details

A. Synopsis

In neocortical explants, derived from developing wild-type and estrogen receptor (ER)-α gene-disrupted (ERKO) mice, it has previously been shown that both 17α- and 17β-estradiol elicit the rapid and sustained phosphorylation and activation of the Mitogen-Activated Protein Kinase (MAPK) isoforms, the Extracellular signal-Regulated Kinases ERK1 and ERK2.

The instant invention demonstrates that the ER mediating activation of the MAPK cascade, a signaling pathway important for cell division, neuronal differentiation and neuronal survival in the developing brain, is neither ER-α nor ER-β, but a novel, plasma-membrane-associated ER with unique properties. The data presented here provide further evidence of the existence of a high-affinity, saturable, ³H-estradiol binding site (Kd ˜1.6 nM) in the plasma membrane. Unlike neocortical ER-α, which is intranuclear and developmentally regulated, and neocortical ER-β, which is intranuclear and expressed throughout life, this functional, plasma membrane-associated ER, designated ER-X, is enriched in caveolar-like microdomains (CLMs) of postnatal, but not adult, wild-type and ERKO neocortical and uterine plasma-membranes.

ER-X, when used in conjunction with the instant invention, means the novel plasma-membrane-associated estrogen receptor characterized herein. The term “ER-X” also appears in the prior art to refer to a postulated, but unidentified, membrane-bound entity (Singh, 1999; Singh, 2000; and Toran-Allerand, 2000).

The ER-X of the instant invention is functionally distinct from ER-α and ER-β and that, like ER-α, it is re-expressed in the adult brain, following ischemic stroke injury. Using a cell-free system described in the experimental methods, ER-α was found to be an inhibitory regulator of ERK activation, as was shown previously in neocortical cultures. Association with CLM complexes positions ER-X uniquely to interact rapidly with kinases of the MAPK cascade and other signaling pathways, providing a novel mechanism for mediation of estrogen's influences on neuronal differentiation, survival and plasticity.

B. Introduction

The traditional view of estrogen action explains inadequately the complete and extensive range of estrogen's effects in the brain, including (i) the very rapid effects of estrogen and (ii) the ability of estrogen to regulate many genes that do not exhibit an apparent estrogen response element (ERE). While such a rapid time course appears inconsistent with transcriptional modulation via classical ERs, it could be explained by the existence of membrane-associated ERs that are coupled to signal transduction pathways typically activated by growth factors.

The existence of plasma membrane-associated ERs has been highly controversial for over 25 years (Pietras, 1977), because of previous failures to isolate and characterize such a membrane-associated receptor protein. Controversy also exists regarding whether membrane ERs represent a subpopulation of classical intranuclear ER-α and ER-β (Blaustein, 1992; Milner, 2001; Razandi, 1999; and Watson, 1999); G-protein-coupled receptors (Benten, 2001; Filardo, 2000; and Kelly, 1999) or novel members of the ER family (Das, 1997; Gu, 1999; and Nadal, 2000).

Most studies propose that membrane-associated ERs are plasma membrane versions of classical ER-α and ER-β (Blaustein, 1992; Milner, 2001; Razandi, 1999; and Watson, 1999). Based on studies of cells transiently transfected with ER-α or ER-β (Razandi, 1999; and Wade, 2001), the prevailing view proposes that both nuclear and plasma-membrane-associated ERs are classical ER-α and ER-β that originate from a single transcript. However, since these cells do not normally express ER-α or ER-β, the extent to which such findings are applicable to estrogen target neurons of the developing CNS is unknown. In contrast, one study suggests that the unoccupied membrane ER may be structurally unique and exhibit intrinsic, ligand-stimulated, tyrosine kinase activity, as do growth factor receptors (Anuradha, 1994; and Karthikeyan, 1996).

The instant invention contributes to (i) advances in the biology of estrogen receptors and of estrogen action in the brain and (ii) pharmacologic intervention and drug development. The selective affinity of 17α-estradiol and other ligands designed to be selective for ER-X and not ER-α would enable prophylaxis and treatment of patients of both sexes for the cognitive deficits and dementias associated with a wide variety of clinical conditions and pulmonary disorders. In addition, ER-X-selective ligands could provide improved treatments for Turner's syndrome and other hypogonadal uterine disorders.

C. Materials and Methods

All animal experiments were conducted in a humane manner, and animals were maintained according to protocols approved by the Institutional Animal Care and Use Committee at Columbia University. ER-X was identified and analyzed by immunoprecipitation, Western blotting and both light and electron microscopy, using cell lysates, detergent-free, highly purified CLM preparations (Smart, 1995), plasma-membranes, post-nuclear supernatants (PNS) and tissue sections obtained from postnatal-day P1-10 and adult wild-type and ER-α gene-disrupted (ERKO) mouse neocortex and uterus.

Mice. Wild-type and ERKO mice were obtained from a breeding colony from matings of C57BL/6J X 129 mice heterozygous (+/−) for the ER-α gene disruption (Lubahn, 1993) and identified by genotyping (Singh, 2000) as either wild-type (+/+) or homozygous (−/−) for the disruption.

Genotyping. Tail snips were obtained from P3-4 pups and used for genotyping, as previously described (Singh, 2000). Briefly, tissues were digested with Proteinase K at 56° C. for 90 minutes, followed by a 99° C. incubation for 10 minutes. The samples were then vortexed vigorously and insoluble material pelleted in a microfuge. Supernatants were used in a PCR reaction that utilized one primer pair (primer 1: 5′-CGG TCT ACG GCC AGT CGG GCA TC-3′; primer 2: 5′-GTA GAA GGC GGG AGG GCC GGT GTC-3′) for the ER-1 gene product (product size=239 base pairs (bp)), and one primer pair (primer 2 from above with NEO Primer: 5′-GCT GAC CGC TTC CTC GTG CTT TAC-3′) for the neomycin insert-containing gene product (product size=790 bp). The PCR program was carried out as follows: 1 cycle at 94° C. for 3 minutes, 30 cycles of 94° C. for 45 seconds, 62° C. for 1 minute, 72° C. for 1 minute 40 seconds, followed by a final extension cycle of 72° C. for 7 minutes. Products were analyzed by agarose gel electrophoresis. Wild-type animals revealed the smaller 239 bp band, homozygous knockouts (ERKO) showed the larger 790 bp band, and heterozygotes displayed both bands.

Neocortical cultures. Organotypic explant cultures, obtained from 360 pm hemi-coronal slices of the frontal and cingulate neocortex of P2 wild-type and ERKO mice (day of birth =P1), were explanted onto collagen-coated, poly-D-lysine pre-coated coverslips and maintained in roller tube culture with gonadal steroid-deficient (gelding serum) and phenol red-free nutrient medium, as previously described (Singh, 1999; and Singh, 2000). The nutrient medium was supplemented with 17β-estradiol (2 nM; Sigma-Aldrich, St. Louis, Mo.) for one week, in order to optimize the development of CNS cultures from estrogen target regions.

Immunoprecipitation and Western blot analysis. Tissues were harvested into protease- and phosphatase-inhibitor-containing lysis buffer (50 mM Tris-base, pH 7.4, 150 mM NaCl, 10% glycerol, 1 mM EGTA, 1 mM Na₃VO₄, 5 μM ZnCl₂, 100 mM NaF, 10 μg/ml aprotinin, 1 μg/ml leupeptin, 1 mM PMSF, 1% Triton X-100) and prepared for immunoprecipitation and polyacrylamide gel electrophoresis, as previously described (Singh, 1999; and Singh, 2000).

Immunoprecipitation was performed, using an indirect technique with magnetic Dynabead® separation (Dynal ASA, Oslo, Norway). All procedures were carried out at 4° C. In brief, P7 wild-type and ERKO cerebral cortices were homogenized by passing the sample eight times through a syringe fitted with a 20-gauge needle. The homogenate was centrifuged at 100,000×g at 4° C. for 15 minutes, and the protein concentration of the supernatant was determined (Lowry's method, Bio-Rad Detergent Compatible Protein Assay Kit®, Bio-Rad®, Hercules, Calif.). For co-immunoprecipitation experiments, detergent was omitted from the lysis buffer. Depending upon the species of the antibodies to be used, the clarified lysates were pre-cleared with either anti-mouse or anti rabbit IgG-coated Dynabeads® to reduce non-specific antibody-antigen binding. For immunoprecipitation of ER-X, the pre-cleared lysates, recovered from the supernatant, were then incubated at 4° C. for 12-24 hours with gentle shaking on a Nutator with 6F11, a mouse monoclonal ER antibody raised against the full-length mouse ER-α molecule, which has proved to be optimal for immunoprecipitation of ER-X (1:50-1:100; Novocastra, Vector Laboratories, Burlingame, Calif.) Primary antibody incubation was followed by the addition of anti-mouse IgG-coated Dynabeads® for 3 hours to capture and precipitate the antibody-antigen complexes. The ER antibodies and co-immunoprecipitated proteins were separated from the Dynabeads by the addition of 1× sample loading buffer, containing 5% β-mercaptoethanol, and boiling for 5 minutes. The Dynabeads® were removed from the supernatant using Dynal Magnetic Particle concentrators. The immunoprecipitated proteins were boiled at 95°-100° C. for 5 minutes, and 300-500 μg samples were loaded onto 10% SDS-PAGE gels and separated based upon molecular size. Prestained rainbow markers (Biorad®, Hercules, Calif.) were used as molecular mass standards. The gels were then electro-blotted onto PVDF membranes.

Immunodetection of the protein of interest was carried out by first blocking the membrane in 5% nonfat dry milk (Carnation) in TBS-Tween (10 mM Tris-base, 150 mM NaCl, 0.2% Tween-20, pH 8.0), followed by addition of the primary antibody. Wherever feasible, the PVDF membranes were probed with antibodies different from those used for immunoprecipitation in order to maximize the specificity of the immunoreactive product obtained. For ER-X in particular, either of two antibodies highly specific for ER-α (one specific for the ligand binding domain (LBD) of ER-α (MC20, 1:500; Santa Cruz Biotechnology, Santa Cruz, Calif.) and the other raised against amino acids 586-600 of the C-terminus of ER-1 (C1355, 1:2000; Upstate Biotechnology (UBI), Lake Placid, N.Y. (Friend, 1997)) were used.

Both antibodies recognize ER-X on Western immunoblots and by immunohistochemistry, but C1355 is not effective for immunoprecipitation. ER-β was identified with antibodies directed against the LBD of ER-β (1:250; Zymed, San Francisco, Calif.). Negative controls to test for the specificity of the interactions were run in parallel and were carried out by immunoprecipitation of the pre-cleared protein lysates with pre-immune mouse IgG and subsequently probed with the appropriate antibody. Additionally, a control peptide or lysate (uterus or ovary) was always used as a positive control to verify the identity of the band in the experimental lanes. The specificity of the signal was determined by the apparent molecular weight (MW) of the protein detected.

Antibody binding to protein was detected using a secondary antibody conjugated to horseradish peroxidase (1:40,000; Pierce Chemical Company, Rockford, Ill.), and visualized autoradiographically on film using enzyme-linked chemiluminescence (ECL®; Amersham Pharmacia Biotech) (Singh et, 1999; and Singh, 2000). All blots were stripped and re-probed with the appropriate antibody to verify equal loading of protein across lanes and were analyzed densitometrically. For studies of ERK phosphorylation, the blots were first probed with phospho-specific ERK antibodies to detect phospho-ERK1/2 (phospho-p44/42 MAP Kinase (Thr202/Tyr204), 1:1000; Cell Signaling, Beverly, Mass.). The same blot was re-probed for total (non-phosphorylated) ERK protein to verify equal loading (ERK-1, C-16, 1:1,000, or ERK-2, C-14, 1:1,000; Santa Cruz Biotechnology). All antibodies were diluted in the blocking solution.

Densitometric analyses. Densitometric analyses of ERK protein levels were performed to ensure similar levels of protein loaded across lanes. Autoradiograms were scanned in triplicate using an HP Scanjet® 6200C (Hewlett Packard Company, Greeley, Colo.) and analyzed using Kodak 1D Image Analysis Software (Eastman Kodak, Rochester N.Y.). Net intensity values were calculated by subtracting the background within the area measured for each band from the total intensity within this same measured area in order to account for any variation in background intensity across the film.

Caveolar-like membrane (CLM) preparation. Membrane fractions were prepared by adapting the detergent-free method of Smart et al. (Smart, 1995). Briefly, pools of 40-50 P7 ERKO neocortices were homogenized in 20 mM Tricine, pH 7.8 buffer, containing 1 mM EDTA, 0.25M sucrose and 1 mM dithiothreitol (TESD buffer), then centrifuged at 1000×g at 4° C. for 10 minutes. The pellet was resuspended in TESD buffer, re-centrifuged, and the supernatants pooled. The combined supernatants were subjected to Percoll® gradient fractionation in the same buffer to isolate the plasma membrane fraction. In some binding experiments (indicated below), Percoll® purified plasma membranes were used without further fractionation. For preparation of CLMs, plasma-membranes were sonicated and further separated by centrifugation on a linear 20% to 10% OptiPrep (iodixanol) gradient (Nycomed Pharma AS, Oslo, Norway). Based upon their light buoyant density, CLMs were separated and purified from non-CLMs using two OptiPrep density gradients. The purity of the CLM preparations was verified immunologically by demonstrating the presence of CLM-enriched proteins: flotillin (1:250, BD Transduction Labs, Lexington, Ky.), PKC-α and PKC-γ (1:1000, BD Transduction Labs), and absence of the non-CLM-associated cytoskeletal protein paxillin (1:10,000, BD Transduction Labs). Electrophoretically separated CLMs on PVDF membranes were probed with antibodies specific for ER-α (C1355, UBI; MC20, Santa Cruz), ER-β (Zymed), flotillin (BD Transduction Labs) and other caveolar-resident proteins (PKC-α and PKC-γ, BD Transduction Labs) and non-caveolar-resident proteins (paxillin, BD Transduction Labs).

Phosphorylation of ERK1/2 in CLM and non-CLM preparations. Phosphorylation of ERK1/2 in ERKO CLM and non-CLM preparations was examined following the method of Liu et al. (Liu, 1997), except that basal medium Eagle (BME) was used in the place of MEM. Nine parts of CLM or non-CLM preparations were mixed with one part of 10×BME, pH 7.4 containing BSA 800 μg/ml, 10 mM NaF, 2 mM Na₃VO₄, leupeptin 100 μg/ml, soybean trypsin inhibitor 100 μg/ml, 10 mM MgCl₂, and 1 mM ATP. For MEK1/2 inhibition of ERK activation, the CLMs were pre-treated with the MEK1/2 inhibitor, U0126 (10 μM; Cell Signaling Technology, Beverly, Mass.) for 30 minutes prior to pulsing them with the appropriate estradiol. Aliquots of ERKO CLMs and non-CLMs were exposed for 30 minutes at 37° C. to either 17α-estradiol (0.1 nM), 17β-estradiol (10 nM), U0126 (10 μM) or a sham control and processed for ERK1/2 phosphorylation, using antibodies to phosphorylated p44/42 MAP Kinase (ERK1/2) (Thr202/Tyr204) (Cell Signaling Technology), as previously described by Singh et al. (Singh, 1999; and Singh, 2000).

Isolation of post-nuclear supernatant (PNS). To increase the yield of ER-X and to test in a cell-free system whether the presence of ER-α is inhibitory for ERK activation, as had been shown previously in neocortical cultures (Singh, 2000), PNS, a cell-free system which contains all the cell organelles except the nucleus, was studied. PNS was isolated from P7 wild-type and ERKO neocortices according to the method of Smart et al. (Smart, 1995). Three to four P7 wild-type and ERKO neocortices were homogenized using a teflon homogenizer in 1 ml of 20 mM Tricine, pH 7.8 buffer, containing 1 mM EDTA, 0.25M sucrose, 10 μg/ml aprotinin and 1 μg/ml leupeptin. The homogenate was centrifuged at 1000×g at 4° C. for 10 minutes. The supernatant obtained is the PNS. The pellet was resuspended in 500 μl of the homogenization buffer, re-centrifuged, and the PNS obtained was pooled with the first PNS. ERKO and wild-type PNS were mixed with 10X phosphorylation buffer, and the MAPK assay was performed as described above. PNS samples were exposed to 17α-estradiol (0.1 nM), 17β-estradiol (10 nM), the ER-α-selective ligand propylpyrazole triol (PPT) (100 nM) (Stauffer, 2000); the MEK inhibitor U0126 (10 μM), BDNF (100 ng/ml); ethanol (0.001%), DMSO (0.001%) and a sham control; first, for 10 minutes at 4° C., followed by 10 minutes at 37° C.

Cholesterol depletion. To determine whether disruption of CLMs impairs estrogen activation of the MAPK cascade, neocortical explants were pre-treated on P9 with the sterol binding agent Nystatin (50 μg/ml) (Sigma-Aldrich), a compound used extensively to document the association of growth factor receptors with caveolae/CLMs (Huang, 1999). This concentration of Nystatin has been shown to result in a significant reduction of cellular cholesterol content without appreciably affecting cell viability (Rothberg, 1990). P9 neocortical explants were exposed to Nystatin (50 μg/ml) (Sigma-Aldrich), BDNF (10 ng/ml) or vehicle control (PBS) for 1 hour prior to pulsing with 10 nM 17β-estradiol for 30 minutes in the continued presence of Nystatin, BDNF or vehicle. Explants were then analyzed by Western immunoblot analysis for phospho-ERK expression using antibodies to phosphorylated p44/42 MAP Kinase (ERK1/2, Thr202/Tyr204; Cell Signaling Technology), as previously described (Singh, 1999; and Singh, 2000).

In situ hybridization. Explants of the ERKO neocortex were processed for in situ hybridization, after 7 days in vitro, by a very sensitive, non-isotopic (digoxigenin) method using a 48 base oligodeoxyribonucleotide (oligonucleotide) to an alpha-specific sequence of the ER-α LBD (BER2), as previously described (Miranda, 1992) Briefly, the probe was 3′-end-labeled with digoxigenin-labeled deoxyuridine triphosphate (dUTP) by terminal deoxynucleotidyl transferase (TdT) (Gibco-BRL, Grand Island, N.Y.). After hybridization of the synthetic oligonucleotide to the target DNA, the hybrids were detected by enzyme-linked immunohistochemistry using anti-digoxigenin antibodies (Fab fragment) conjugated to alkaline phosphatase (1:500; Boehringer-Manheim, Indianapolis, Ind.), and an enzyme-catalyzed blue-color reaction (5-bromo-4-chloro-3-indolyl phosphate and nitro-blue tetrazolium salt).

Immunocytochemistry. P7 ERKO and wild-type mice were anesthetized by hypothermia and killed painlessly by transcardial perfusion of saline, followed by 4% paraformaldehyde and 1% glutaraldehyde fixation. The neocortex was processed by pre- and post-embedding immunocytochemistry for ER-α and flotillin, respectively. Sections (50 μm) were incubated in anti-ER-α antibodies (C1355, 1:1000; or 6F11, 1:50), washed and incubated in biotinylated horse-anti-rabbit or anti-mouse IgG (1:250; Vector), incubated with avidin-biotin-peroxidase (1:50; Vector), and followed by diaminobenzidine (DAB) (brown reaction product). Sections were then processed for electron microscopy, dehydrated and flat embedded in Durcupan® (EM Science, Gibbstown, N.J.). Alternate ultrathin sections (Reichert-Jung Ultramicrotome) of the neocortex, immunolabeled for ER-α, were further labeled for flotillin (1:50). Sections were washed and incubated in gold-conjugated (15 nm) goat anti-rabbit IgG (1:20; EM Science) then washed and contrasted with saturated uranyl acetate. Ultrathin sections were examined using a Philips CM-10 electron microscope.

Estrogen binding assay. Duplicate aliquots of 1 mg each of protein lysate from ERKO P7 neocortex or wild-type adult uterus were pre-cleared for 30 minutes using anti-rabbit-IgG-coated magnetic beads (Dynal AS). Pre-cleared protein lysates were immunoprecipitated with anti-ER-α antibodies (6F11, Novocastra; or MC20, Santa Cruz) at 4° C. overnight. Immunoprecipitated samples, Percoll®-purified plasma membrane fractions and Optiprep-purified CLM preparations (50 μg each) from P7 wild type or ERKO neocortex were incubated with ³H-estradiol (2, 4, 6, 7, 16, 17-³H estradiol, 100 Ci/mmol; NEN Life Sciences, Boston, Mass.) at 4° C. for 18 hours. The incubation was terminated by adsorption of the binding sites onto an equal volume of hydroxylapatite (HAP) slurry in TESD buffer. HAP pellets were washed four times with Tris-buffered saline containing 0.2% Tween-20 buffer and extracted with 1 ml absolute ethanol overnight at room temperature. The ethanol supernatants were transferred to liquid scintillation fluid (5 ml) and counted. Control tubes, used in assessing HAP adsorption of free steroid, contained HAP and the same buffer constituents, without addition of the membranes. Non-specific binding was assayed in the membranes using the same amount of radioactive ligand plus 200-fold molar excess of unlabeled diethylstilbestrol (DES) (Sigma-Aldrich). Specific binding was calculated by subtracting non-specific from total binding. The apparent affinity of the membrane binding sites was determined by incubation with a range of concentrations of ³H-estradiol (0.25-10 nM). The specificity of the binding sites was studied by co-incubation of purified membranes with 2 nM ³H-estradiol in the presence of unlabeled progesterone, 17α-estradiol or 17β-estradiol, added at either 25-fold or 500-fold Molar excess.

Transient cerebral ischemia model. Details of the murine model of focal cerebral ischemia, using an intraluminal suture, have been described previously (Huang, 2000). Briefly, mice were anesthetized with 0.3 ml of intraperitoneal ketamine (10 mg/ml) and xylazine (0.5 mg/ml) and positioned supine on a rectal temperature-controlled operating surface (Yellow Springs Instruments, Yellow Springs, Ohio). Animal core temperature was maintained at 37+/−2° C. during surgery and for 90 minutes after surgery. A midline neck incision exposed the right carotid sheath under the operating microscope (Leica®). The common carotid artery was isolated and the occipital, pterygopalatine, and external carotid arteries were each isolated, cauterized and divided. Middle cerebral artery occlusion was accomplished by advancing a 13-mm heat-blunted 6.0 nylon suture via an arteriotomy made in the external carotid stump. After placement of the occluding suture, the external carotid artery was cauterized to prevent bleeding through the arteriotomy, and arterial flow was established. After 45 minutes the occluding suture was removed, and electrocautery was used to close the arteriotomy. The wound was closed with surgical staples. After 24 hours, the mice were anesthetized, decapitated, and brains were removed intact and placed in a mouse brain matrix (Activational Systems Inc, Warren, Mich.) for 1 mm sectioning. Sections were immersed in 2% triphenyltetrazolium chloride (Sigma-Aldrich) in 0.9% saline and incubated for 12 minutes at 37° C. Infarcted brain was identified as an area of unstained tissue. Slices containing tissue from the region surrounding the infarct (penumbra) and from the comparable region of the non-infarcted hemisphere were processed for immunoprecipitation and Western analysis, using 6F11 and MC20 antibodies to ER-α, respectively. A total of 8 wild-type mice were studied.

D. Results

P7 Neocortex Contains a ˜62-63 kDa Protein that is Neither ER-α nor ER-β and Which is Enriched in CLMs of the Plasma Membrane

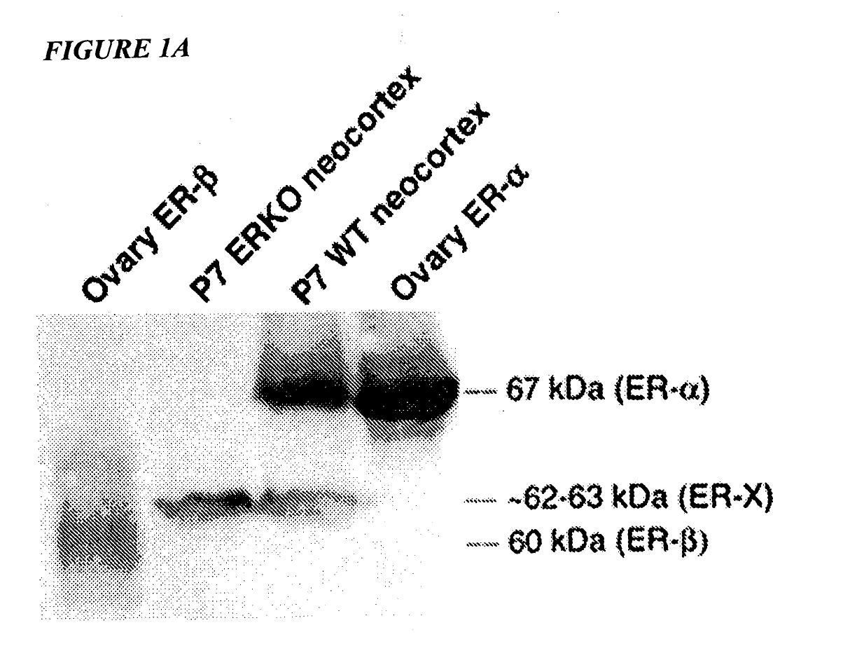

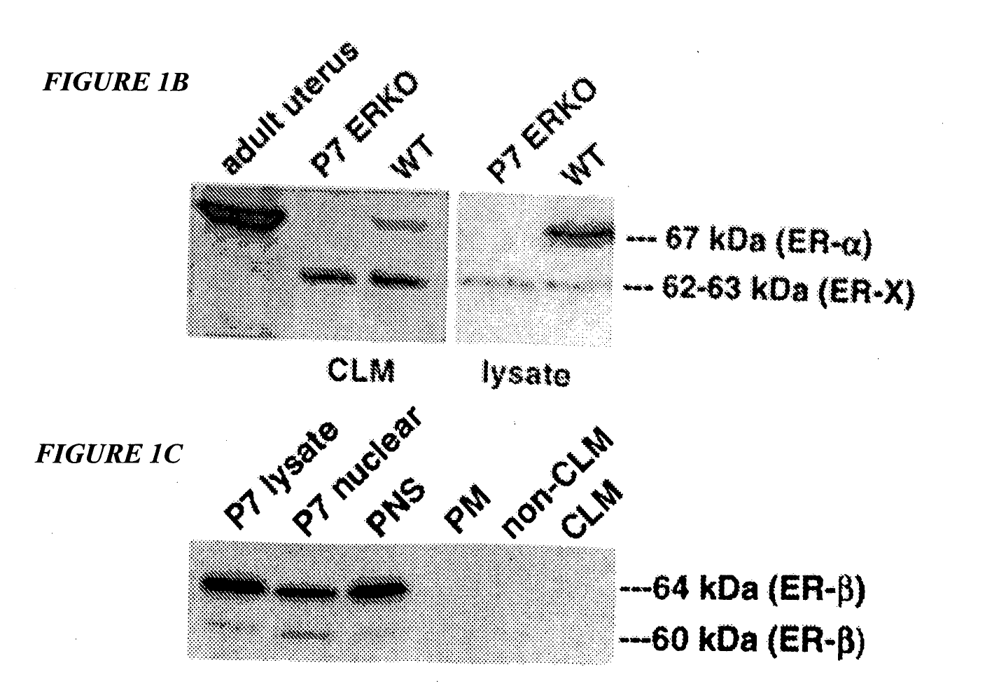

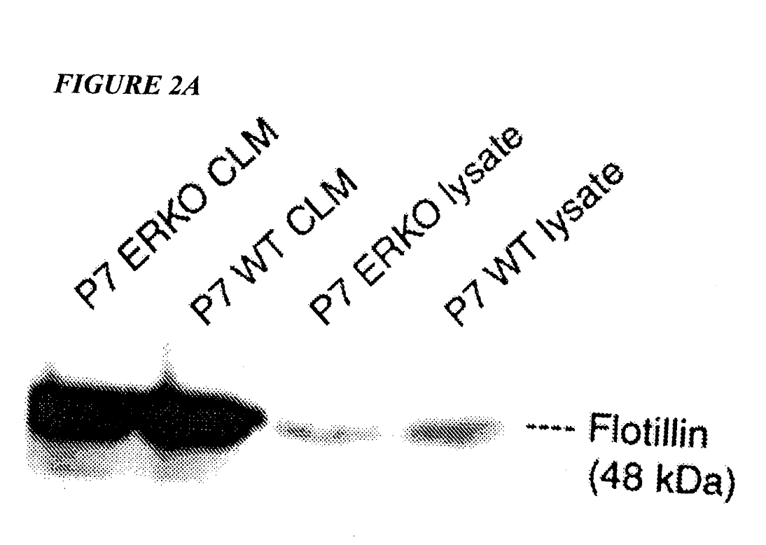

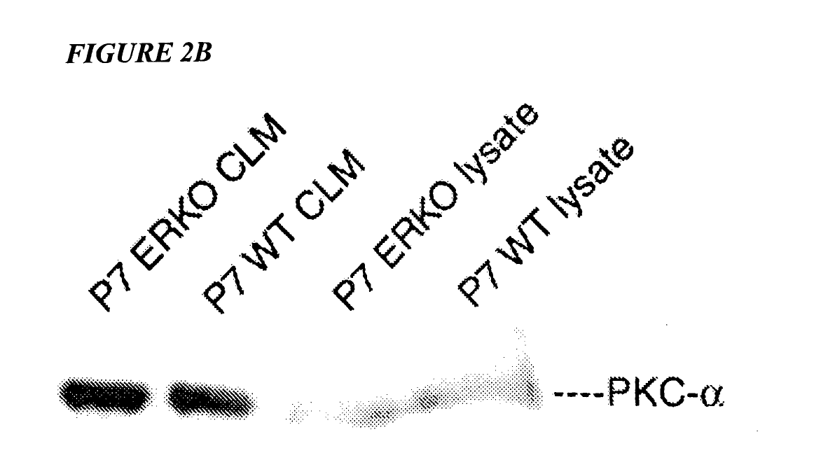

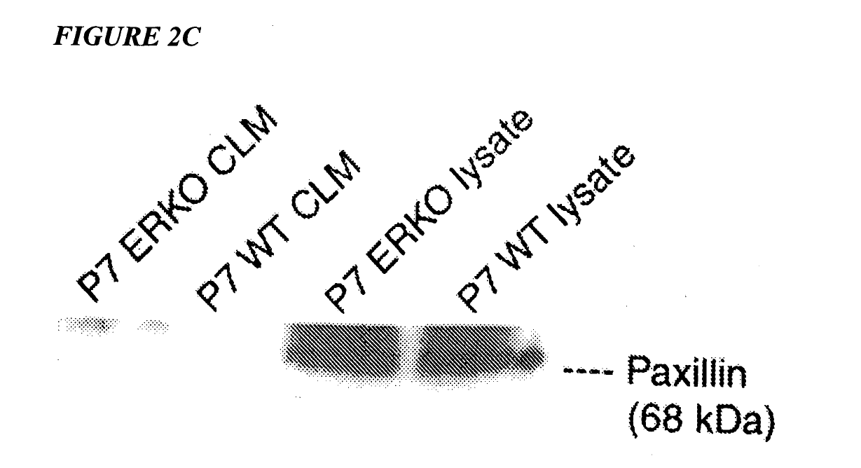

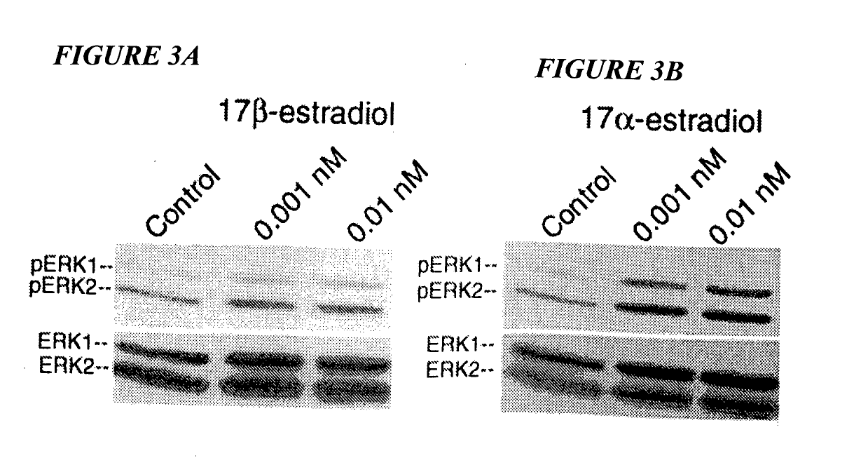

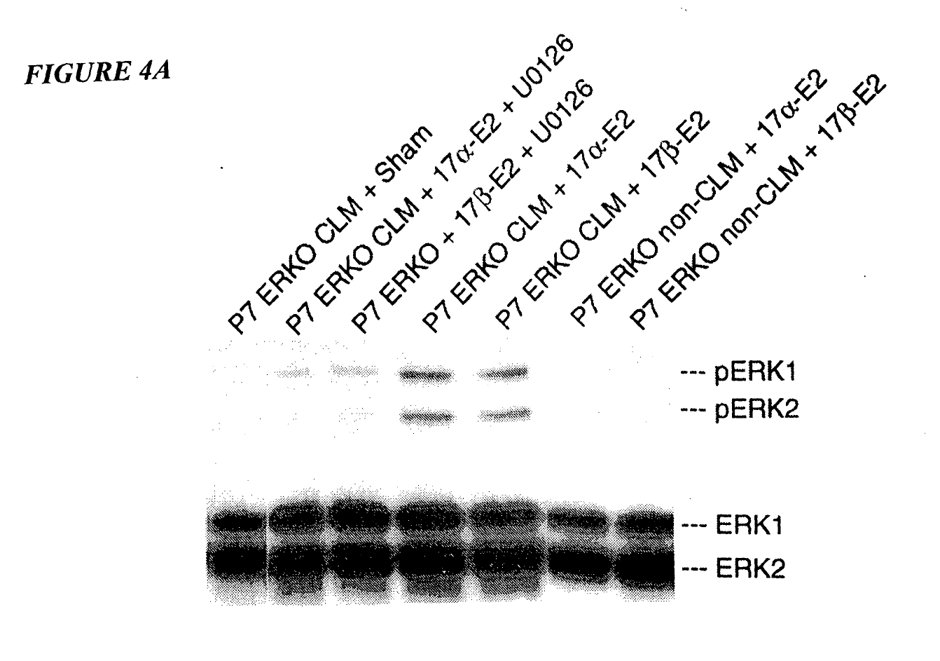

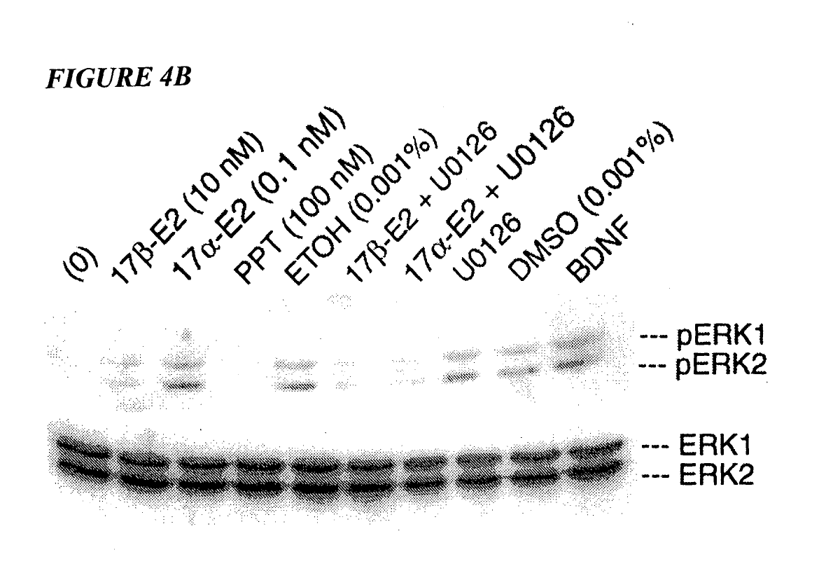

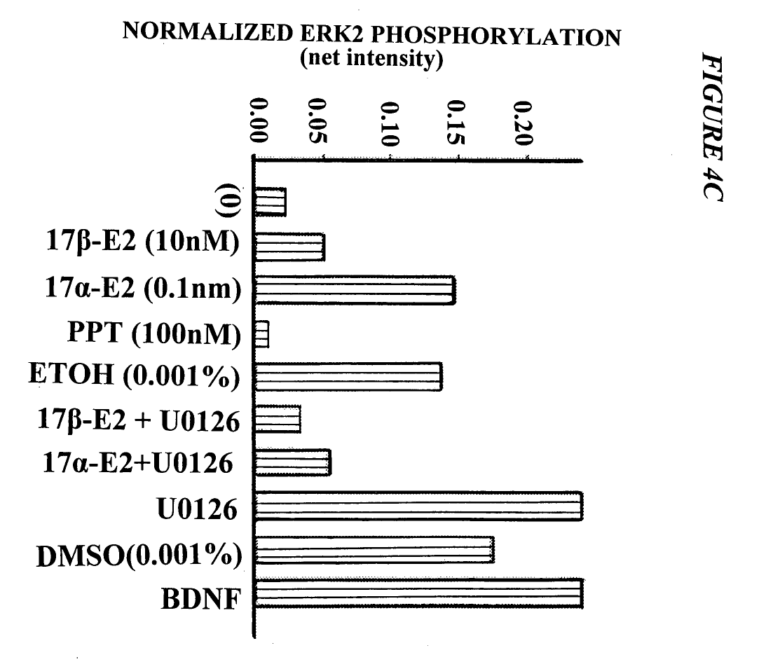

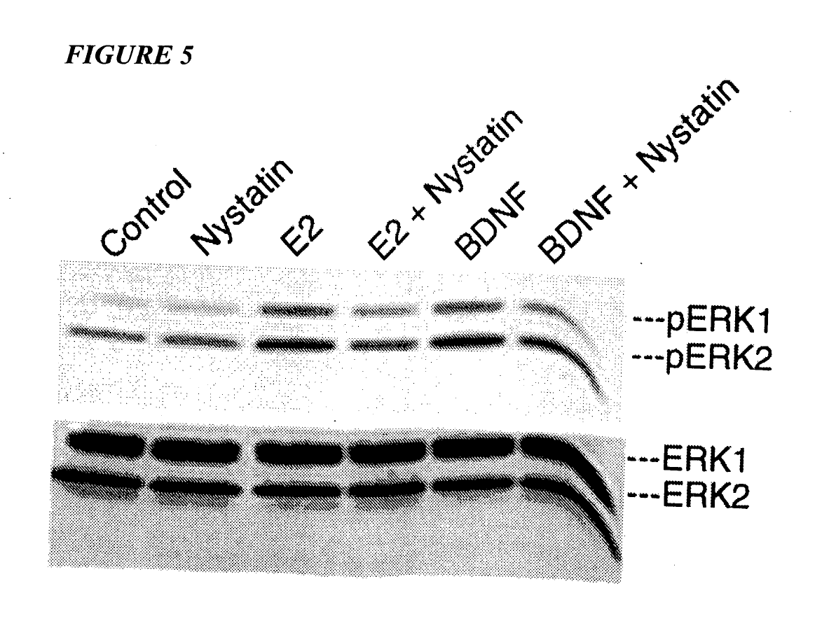

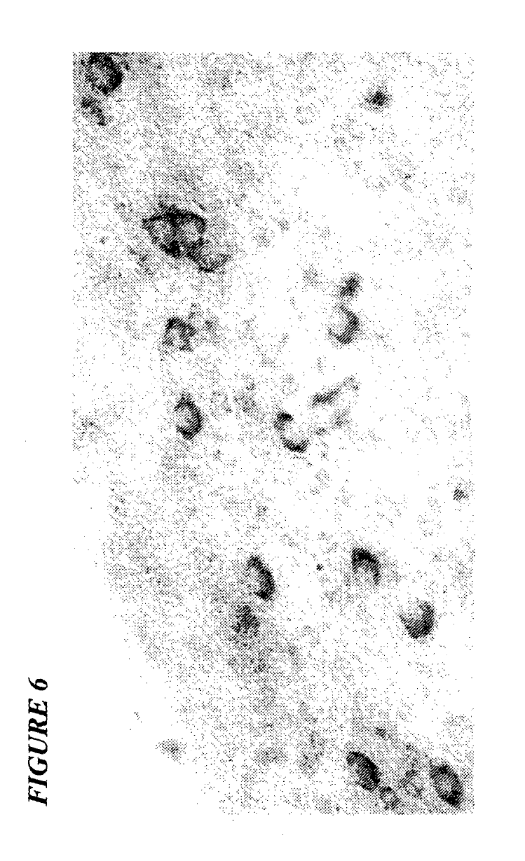

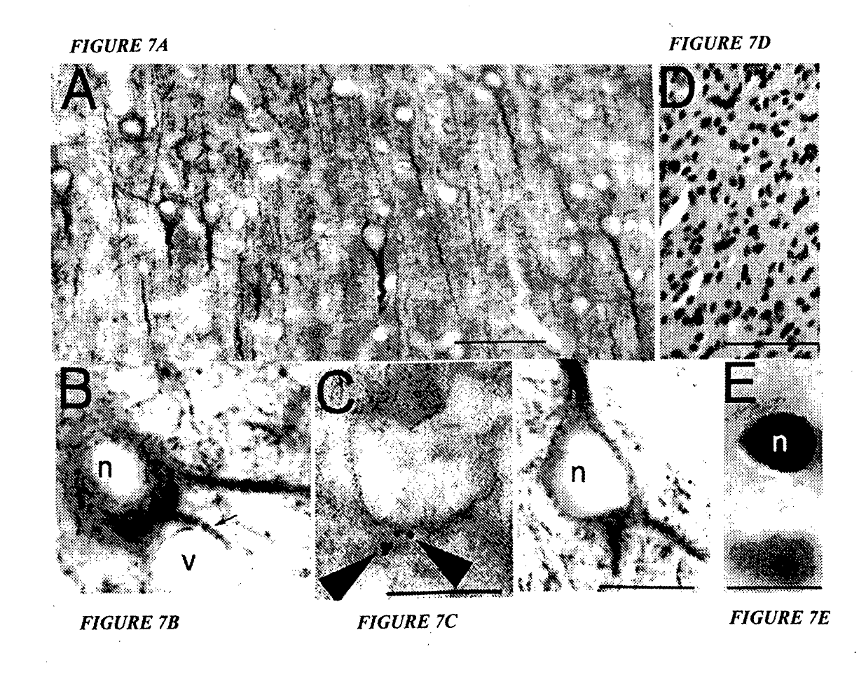

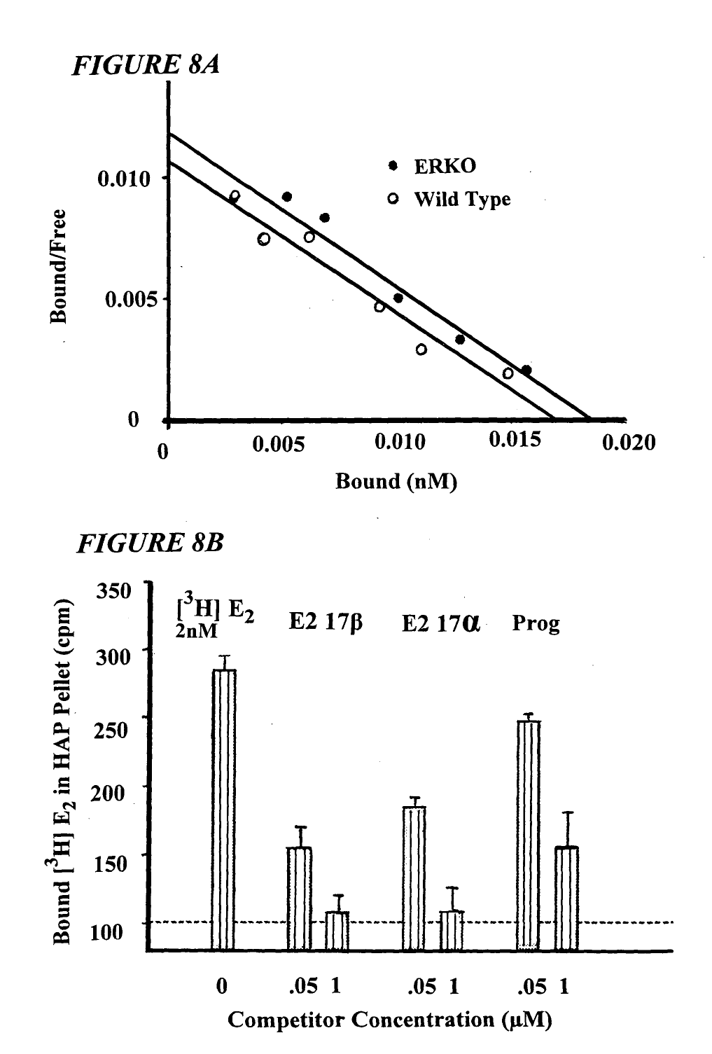

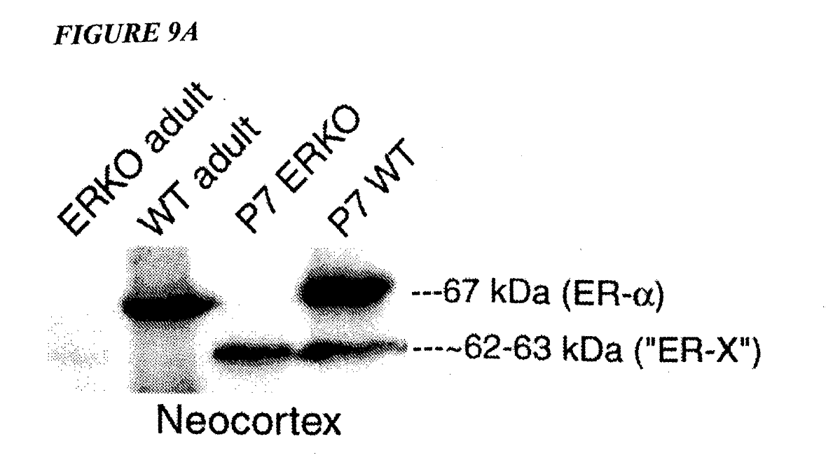

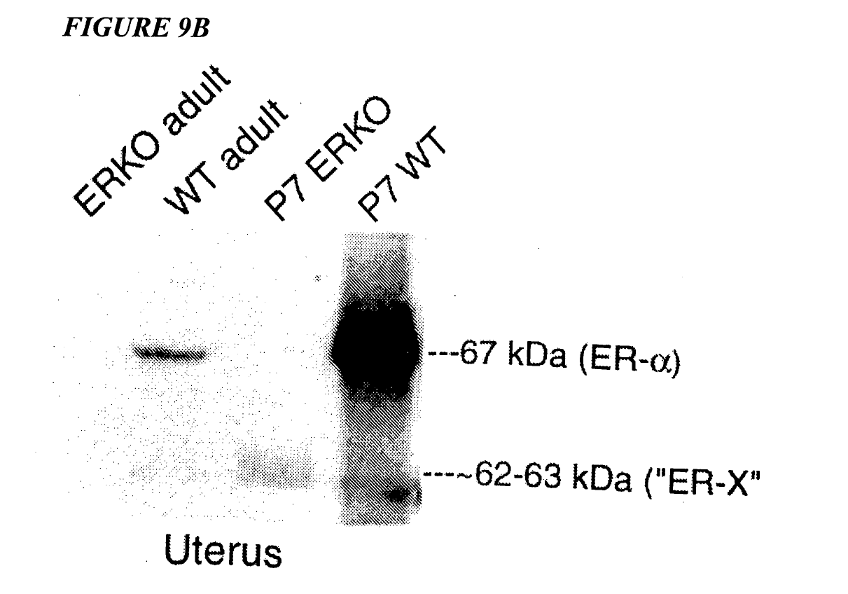

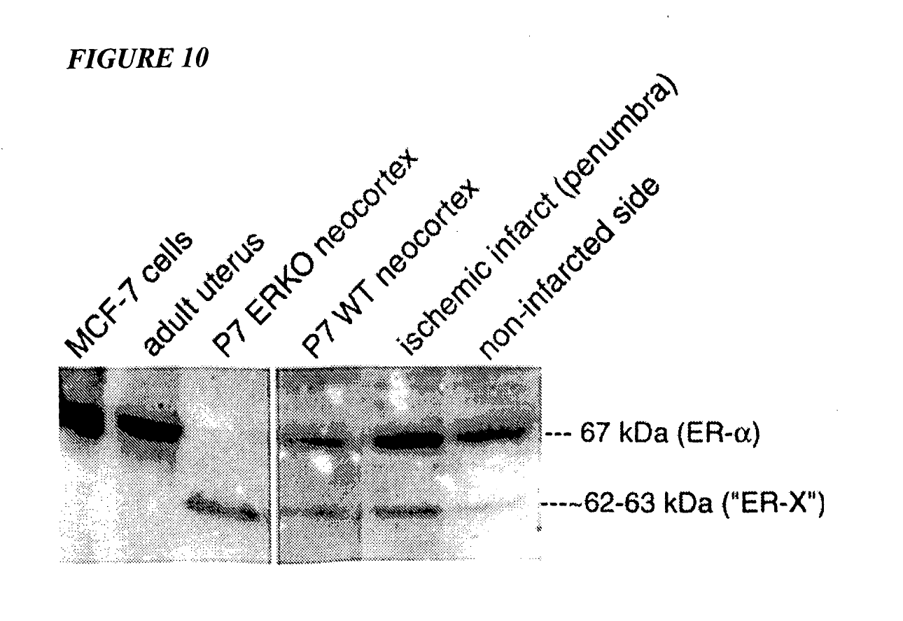

A previously unknown protein was identified in wild type and ERKO P7 neocortical cell lysates, postnuclear supernatant (PNS) and caveolar-like membrane (CLM) preparations, by immunoprecipitation and Western immunoblot analysis using antibodies directed against ER-α and ER-β. This protein is immunoreactive for the ligand-binding domain (LBD) of ER-α but not ER-β. Although immunoreactive for ER-α, this protein has an apparent MW of ˜62-63 kDa that is clearly different from that of ovarian ER-α (67 kDa) and ER-β (60 kDa) (Fitzpatrick, 1999). This new protein has been designated “ER-X” in keeping with the nomenclature used earlier regarding a postulated membrane-bound entity (FIG. 1a). Cell lysates and detergent-free, highly purified, CLM preparations (Smart, 1995) of both P7 neocortical wild-type and ERKO plasma-membranes expressed this ˜62-63 kDa protein (FIG. 1b). While P7 wild-type neocortex expressed both the 67 kDa ER-α band and the ˜62-63 kDa ER-X band, P7 ERKO neocortex contained only the ˜62-63 kDa band. P7 wild-type and ERKO neocortical CLM preparations were greatly enriched with the ˜62-63 kDa protein (FIG. 1b). A striking reversal of the 67 kDa/˜62-63 kDa ratio was seen in wild-type P7 neocortical CLM preparations, which, while highly enriched in the ˜62-63 kDa form, were greatly diminished in the 67 kDa ER-α band. The specificity and significance of the association of the ˜62-63 kDa protein with CLMs was emphasized by the failure to detect immunoreactivity for other steroid receptors, such as ER-β in CLM, non-CLM and plasma membrane preparations (FIG. 1c), although its presence was clearly demonstrable in P7 neocortical cell lysates and in the nuclear fraction and PNS (FIG. 1c).

The purity of the CLM preparations was verified by demonstrating the presence of the CLM integral protein flotillin (Bickel, 1997) (FIG. 2a) and such CLM-enriched resident proteins as PKC-α (FIG. 2b) and PKC-γ (data not shown) (Smart, 1995), and by the absence of the cytosolic protein paxillin (FIG. 2c), a cytoskeletal component associated with non-CLM regions of plasma-membranes (Smart, 1995).

ER-X is Also Expressed in Other Cell Types and Tissues

Using the same methodology described above, different cell types and tissues were analyzed for the presence of ER-X. ER-X was detected in brain tissue samples isolated from postnatal rat and fetal baboon, in lung tissue samples isolated from fetal baboon, and in cell extracts prepared from Saccharomyces cerevisiae and mouse stem cells. In each sample, the approximate molecular weight of the immunoreactive band was 62-63 kDa (data not shown).

ER-X has an Entirely Different Steroid Specificity than Either ER-α or ER-β

The steroid specificity for estrogen-induced activation of ERK1/2 phosphorylation is radically different from that of either ER-α or ER-β. ERK1/2 is not activated by either ER-α-selective ligands such as 16α-iodo-17β-estradiol (Singh, 2000) and propylpyrazole triol (PPT) (100 nM) (Stauffer, 2000) (FIGS. 4b and 4c) or by ER-β-selective ligands such as genistein and coumestrol (Singh, 2000), but is activated equally well by picoMolar concentrations of 17α-estradiol and 17β-estradiol (FIGS. 3a and 3b). In wild-type cultures 17α-estradiol, a natural stereoisomer of 17β-estradiol that is generally considered to be transcriptionally inactive, elicited a stronger, sustained activation of ERK1/2 at the 1-10 pM (10⁻¹² M) range (FIG. 3b) than did 17β-estradiol (0.1-10 nM) (FIG. 3a). What makes this response so astonishing is that 17α-estradiol, which, like 17β-estradiol, is derived from aromatization of androgens, but whose site of synthesis is unclear, has a 100-fold lower affinity for ER-α than 17β-estradiol (Hajek, 1997). Significantly, higher levels of 17β-estradiol were required for ERK activation in wild-type neocortical cultures (FIG. 3a), perhaps reflecting the need to overcome the inhibitory effect of ER-α on ERK1/2 phosphorylation (Singh, 2000) (FIG. 4), which, unlike 17α-estradiol, 17β-estradiol activates as well. That the inhibitory presence of ER-α influences dose-responsiveness is suggested by observation that in the ER-α-deficient ERKO neocortical explants, 17β-estradiol, like 17α-estradiol, is also able to elicit activation of ERK in the 1-10 picoMolar range (data not shown).

Estrogen Elicits ERK1/2 Activation in CLMs

To provide direct evidence that the CLM-associated ˜62-63 kDa ER-X protein is connected with estrogen-induced ERK1/2 activation, it was demonstrated that exposure of highly purified P7 ERKO neocortical CLMs to 17β-estradiol (10 nM) and 17α-estradiol (0.1 nM) for 30 minutes elicited phosphorylation of ERK1/2 (FIG. 4a). In both instances ERK activation was inhibited by the MEK inhibitor U0126 (FIG. 4a). In contrast, non-CLM regions of the plasma membrane, exposed similarly, did not respond (FIG. 4a).

ER-α is an Inhibitory Regulator of ERK1/2 Activation in PNS

Wild-type PNS, a cell-free system, was used to test whether ER-α is an inhibitory regulator of estrogen-induced ERK1/2 activation, which had been previously shown in neocortical explants (Singh, 2000). Using the ER-α-selective ligand PPT (100 nM) (Stauffer, 2000) in wild-type neocortical PNS resulted in a dramatic reduction in MEK-inducible ERK1/2 phosphorylation to below baseline (FIG. 4b). Of particular note, furthermore, were the findings that the levels of 17β-estradiol-induced ERK1/2 phosphorylation were significantly less than the levels following exposure to 17α-estradiol, although both were inhibited by the MEK inhibitor U0126. This difference in responsiveness may be attributed to the fact that, at P7, wild-type neocortex is enriched with maximal levels of ER-α (Gerlach, 1983) which, when activated by 17β-estradiol (but not 17α-estradiol), exert an inhibitory effect on ERK1/2, as is also seen following exposure to PPT. These findings confirm that ER-α is a strong inhibitor of ERK1/2 activation, a measure of which is given by the ability of PPT to effectively prevent activation of ERK1/2 even in the face of strong ERK1/2 activation, elicited by the PPT vehicle ethanol (FIGS. 4b and 4c). These findings provide not only proof that ER-α does not mediate activation of the MAPK cascade but also compelling evidence confirming the role of ER-α as an inhibitory modulator of ERK1/2 activation.

Cholesterol Disruption in CLMS Decreases Estrogen Activation of ERK

CLMs, like caveolae, are highly enriched in cholesterol, glycosphingolipids, sphingomyelin and lipid-anchored membrane proteins, which serve as multi-valent scaffolding onto which many signaling kinases assemble to generate pre-assembled signaling complexes. Eighty to ninety percent of plasma membrane cholesterol is concentrated within caveolae/CLMs, where it plays a critical role in maintaining receptor protein association within the CLM domain (Rothberg, 1990). The sterol binding-agent Nystatin has been used extensively to document the association of growth factor receptors with caveolae/CLMs (Huang, 1999). To determine whether selective disruption of cholesterol in CLMs impairs the ability of estrogen to elicit ERK1/2 phosphorylation, P9 neocortical explants were exposed to Nystatin (50 μg/ml) for 1 hour prior to pulsing with 17β-estradiol (10 nM), BDNF (100 ng/ml) or the vehicle control (PBS) for 30 minutes (FIG. 5) and then measuring ERK1/2 phosphorylation by Western blot analysis. Disruption of membrane cholesterol decreased the ability of both estradiol and BDNF to elicit ERK1/2 phosphorylation, providing additional evidence of the contributions of CLMs to estradiol-induced ERK1/2 activation.

ER-X has Homology with ER-α LBD and is Expressed in the Plasma Membrane

Using an oligonucleotide probe directed against an alpha-specific region of the ER-α LBD (BER2) (Miranda, 1992), widespread distribution of the blue ER-α-like hybridization signal was found in neurons of cultured slices of the ER-α-deficient P2 ERKO neocortex, 17 days in vitro (FIG. 6). This pattern of hybridization in ERKO neocortex suggests that, in view of the absence of ER-α, the oligonucleotide sequence used may share some homology with ER-X mRNA.

Direct evidence that ER-X may be a neuronal plasma-membrane-associated ER protein with some homology to ER-α was also obtained in the ERKO neocortex by means of light and electron microscopic immunohistochemistry (FIGS. 7a to 7e). Using polyclonal antibodies generated against the final 14 C-terminal amino acids of the rat ER and highly specific for ER-α (C1355, UBI) (Schreihofer, 1999), large numbers of immature ERKO neocortical neurons with unstained nuclei were seen (FIGS. 7a and 7b). Immunoreactivity was clearly localized to the cell membrane and cytoplasm and not in the nucleus. In FIG. 7b, a blood vessel (V) is in close proximity to a labeled dendrite, an association which suggests a mechanism by which estrogen could get even more efficiently onto ER-X. On the other hand, using monoclonal antibodies generated against full-length mouse ER-α, (6F11, Novocastra) (FIGS. 7d and 7e) which have been reported to recognize the 5′ N-terminus region, the opposite result was obtained: nuclear labeling was observed but no cytoplasmic or membrane labeling was seen. Since 6F11 cross-reacts significantly with ER-β by Western blotting (data not shown), the nuclear labeling observed here most likely reflects intranuclear ER-β which is normally expressed in both wild-type and ERKO neocortex. Association of the ˜62-63 kDa protein with CLMs was further documented at the ultrastructural level on ultrathin cryostat sections of P7 ERKO neocortex by demonstrating immunoreactive flotillin, labeled by gold particles, co-localized with horseradish peroxidase-labeled immunoreactivity for ER-α on a neocortical dendritic spine (FIG. 7c).

ERKO Neocortical Plasma-Membranes Contain an Estrogen-Binding Protein (ER-X)

It was determined that neocortical plasma-membranes contain a unique estrogen-binding protein by scintillation counting of ³H-estradiol binding to the ˜62-63 kDa ER-X protein in highly purified P7 ERKO CLM preparations. In these preparations, the only detectable ER immunoreactive material present was the ˜62-63 kDa protein (FIG. 1). Binding of 10 nM ³H-estradiol to P7 ERKO CLMs appeared to be specific and saturable, in that it was suppressed in the presence of unlabeled diethylstilbestrol (DES). Neocortical CLM preparations from P7 ERKO mice, shown to be highly enriched in ER-X, were similarly highly enriched in DES-sensitive estrogen binding (282.12 fmol/mg CLM protein), as compared to P7 ERKO neocortical lysates (9.94 fmol/mg lysate protein) and wild-type adult uterine lysates (38.85 fmol/mg lysate protein). Further characterization of the membrane binding sites was achieved using Percoll®-fractionated plasma-membranes, containing both CLM and non-CLM components, to increase the yield of total membrane sufficiently to allow construction of binding isotherms and performance of specificity studies. In Percoll®-purified membranes from P7 ERKO neocortices, as in CLMS, the only detectable ER immunoreactive protein present was the ˜62-63 kDa band (data not shown). Membranes from both P7 ERKO and P7 wild type neocortex contained a high-affinity, saturable ³H-estradiol binding site (Kd ˜1.6 nM; FIG. 8a). Addition of 50 nM unlabelled 17β-estradiol or 17α-estradiol markedly inhibited binding of ³H-estradiol. In the presence of a 1 μM concentration of either estrogen, binding of the tritiated ligand was reduced to the non-specific levels observed in the presence of excess DES (FIG. 8b). Unlabelled progesterone, by contrast, was less effective than either estrogen, progesterone only partially suppressing binding of ³H-estradiol when added in 500-fold molar excess (FIG. 8b).

ER-X is Developmentally Regulated in the Brain and Uterus

Expression of the ˜62-63 kDa ER-X protein is developmentally regulated and is maximally expressed ˜P7-10 in both the neocortex and uterus (FIGS. 9a and 9b). During the first postnatal month, wild-type and ERKO neocortical and uterine levels of the ˜62-63 kDa protein declined until P21 and became dramatically reduced in the adult, which expressed little of this protein.

ER-X is Up-Regulated in a Rodent Model of Brain Injury

To test whether re-expression of the developmentally regulated ER-X might return following brain injury in the adult, as has been reported for the developmentally regulated ER-α (Dubal, 2001), a mouse ischemic stroke model, elicited by transient intraluminal middle cerebral artery occlusion, was used (Huang, 2000). Tissue from the region surrounding the infarct (the penumbra) was compared with the comparable region of the non-infarcted neocortex of the opposite side, 24 hours following occlusion. Using immunoprecipitation, followed by Western blotting, the ˜62-63 kDa protein was upregulated in the penumbra (FIG. 10) to levels comparable to those present during development, as compared to the non-infarcted side which remained unchanged. There was also up-regulation of ER-α (FIG. 10), as has been shown previously (Dubal, 2001).

ER-X is Up-Regulated in a Rodent Model of Alzheimer's

Expression levels of ER-X in the neocortex and hippocampus of wild-type and Alzheimer's disease model transgenic mice were measured by Western Blot analysis. ER-X expression levels were upregulated in aging wild-type mice as compared to young adult wild-type mice who expressed little if any ER-X. ER-X expression levels were also found to be significantly higher in Alzheimer's disease model transgenic mice exhibiting advanced Alzheimer's disease characteristics as compared to those exhibiting early Alzheimer's disease characteristics. In each comparison above, ER-α expression was also upregulated but to a significantly lesser degree than ER-X (data not shown).

E. Discussion

These data point strongly to the existence of a novel, plasma-membrane-associated, estrogen receptor (ER-X). Although membrane ERs have been identified immunologically as ER-α in several cell and tissue systems (Blaustein, 1992; Milner, 2001; Razandi, 1999; and Watson, 1999), the instant invention demonstrates that ER-X is a unique, functionally distinct, and hitherto unidentified receptor, based upon its MW, ligand specificity, cellular localization and apparent response characteristics (see Table 1 for comparisons).

TABLE 1
Characteristics of estrogen receptors

| | ER-α | ER-β | ER-X |
|---|---|---|---|
| Molecular | 67 kDa | 60 kDa | 62-63 kDa |
| Weight | | | |
| Cellular | Intranuclear | Intranuclear | Plasma membrane |
| localization | | | |
| Selective | 16α-iodo-17β- | Genistein and | 17α-estradiol |
| Ligand* | estradiol and | coumestrol | |
| | Propylpyrazole | | |
| | triol (PPT) | | |
| Stereospecific | Yes | Yes | No |
| binding of | 17β-estradiol > | 17β-estradiol > | 17β-estradiol ≅ |
| estradiol | 17α-estradiol | 17α-estradiol | 17α-estradiol |
| Regulation of | Developmentally | Constitutively | Developmentally |
| Expression | regulated | expressed | regulated |
| Immunoreactive | Yes | No | Yes |
| with MC20 | | | |
| antibody | | | |
| Effect on MAPK | Inhibits | No Effect | Activates |
| pathway | | | |
| activation | | | |

*“Selective ligand” means a ligand which binds to the indicated receptor either exclusively or with a much greater affinity than that with which it binds to the other two receptors.

Although ER-X reacts with antibodies to the ER-α LBD, ER-X is not membrane-associated ER-α. The MW of ER-X (˜62-63 kDa) is clearly different from that of both ER-α (67 kDa) and ER-β (60 kDa) (FIG. 1a). While a functional isoform of ER-β with an additional 18 amino acids inserted in the LBD has been identified in rat and mouse tissues (ER-β2) (Peterson, 1998), ER-X cannot represent ER-β2, because (i) antibodies directed against the ER-α LBD cross-react with ER-X and do not recognize intranuclear ER-β; (ii) no immunoreactivity was detected in blots from CLMs enriched in ER-X using the anti-ER-β antibody (Zymed), which does not react with ER-α but does cross-react on Western blots with the molecular isoforms of rat ER-β observed in tissue lysates; (iii) ERK1/2 is not activated by ER-α or ER-β-selective agonists (Singh, 2000) (FIG. 4b); and (iv) unlike ER-α or ER-β, ER-X is not stereo-specific, responding equally well to picomolar concentrations of 17α-estradiol and 17β-estradiol (FIGS. 3a and 3b), while ER-α and ER-β exhibit a markedly higher affinity for 17β-estradiol than for 17α-estradiol (Kuiper, 1997).

ER-X is part of a multi-molecular CLM complex, comprising immunoreactivity for ER-α (but not ER-β) in association with hsp90, members of the MAPK cascade (Singh, 1999; Toran-Allerand, 1999; and Toran-Allerand, 2000) and flotillin, the multi-valent, 48 kDa scaffolding protein and neuronal homologue of the caveolar protein caveolin (Bickel, 1997). Two recent studies (Levin, 2002; and Razandi, 2002) report association of ER-α immunoreactivity with caveolae in vascular and breast cancer (MCF-7) cells. While caveolin-associated ER was identified by the authors as ER-α. (Razandi, 2002), the MW of the immunoreactive band was stated to be 62 kDa, not 67 kDa, as would be expected for authentic full-length ER-α. In vascular and MCF-7 cells, like neuronal CLMs, caveolar-associated ER-α immunoreactivity represents primarily a protein with an apparent MW approximately 5 kDa less than that of authentic ER-α. In brain, both P7 wild-type and ERKO neocortical CLM preparations were greatly enriched with the immunoreactive ˜62-63 kDa ER-X protein (FIG. 1b) and depleted of ER-α and ER-β (FIGS. 1b and 1c), supporting the selectivity and specificity of the ER-X association with CLMs.

Surprisingly, in both wild-type and ERKO neocortical explants and CLMs, 17α-estradiol, the natural stereoisomer of 17β-estradiol with 100-fold lower affinity for ER-α, (Hajek, 1997) also elicited sustained MEK-dependent activation of ERK1/2 in the picoMolar range (FIGS. 3b, 4a, and 4b). ER-α-selective and ER-β-selective ligands fail to elicit ERK1/2 activation in wild-type neocortical explants and ER-α may even be an inhibitory regulator of ERK activation (Singh, 2000). This has been confirmed in the PNS cell-free system (FIGS. 4b and 4c). The absence of an inhibitory response in ERKO PNS (FIG. 4c) is consistent with the absence of authentic 67 kDa ER-α from ERKO brains.

Nystatin disrupts cholesterol in cell membranes (Iwabuchi, 2000) by forming globular deposits that alter the planar organization of the membrane (McGookey, 1983), thereby selectively inhibiting caveolar trafficking without altering other cell functions such clathrin-mediated endocytosis (Ros-Baro, 2001) or intracellular receptor trafficking back to the cell surface (Subtil, 1999). Nystatin (50 μg/ml) has been shown to significantly reduce cellular cholesterol content without appreciably affecting cell viability. This concentration of Nystatin impaired estradiol induced ERK1/2 activation (FIG. 5).

The existence of plasma membrane-associated ERs (Pietras, 1977) has been controversial because of previous failures to isolate and characterize such a membrane-associated receptor. Hypothetical mechanisms have included plasma-membrane versions of classical intranuclear ER-α and ER-β (Blaustein, 1992; Milner, 2001; Razandi, 1999; and Watson, 1999), novel members of the ER family (Das, 1997; Gu, 1999; and Nadal, 2000); G-protein-coupled receptors (Filardo, 2000; Kelly, 1999; and Wyckoff, 2001); or even growth factor-like receptor tyrosine kinases (Anuradha, 1994).

That ER-X may have sequence homology with the ER-α LBD is suggested by (i) the strong hybridization signal obtained in ERKO neocortical explants with an oligonucleotide probe specific for the ER-α LBD (Miranda, 1992) (FIG. 6) and (ii) ER-α-like immunoreactivity in ERKO neocortex, using antibodies to the ER-α LBD (FIGS. 7a and 7b) but not with those recognizing the N-terminal region (FIGS. 7d and 7e). In order to generate ERKO, the ER-α gene was disrupted by insertion of a 1.8 kb PGK—Neomycin sequence in the region of exon 2, approximately 280 bp downstream of the transcription start codon (N-terminus) (Lubahn, 1993), a region far upstream from the LBD (exons 4-8). Therefore, ER-α-like mRNA found in ERKO neocortex may represent (i) residual, untranslated ER-α mRNA; (ii) a splice variant of ER-α; or (iii) ER-X mRNA itself. Residual, weak estrogen binding not attributable to ER-β has been reported in both ER-α (ERKO) and ER-α/ER-β (double) knockout adult mouse brains (Shughrue, 2002). This binding was identified in ERKO only as a splice variant of ER-α at exon 2 that may regulate the progesterone receptor. Nonetheless, there are compelling reasons that ER-X does not represent the protein product of such a splice variant. A splice variant at exon 2 would contain exactly the same LBD sequence as authentic ER-α. However, the ligand specificity of ER-X is clearly different from that of ER-α in that ER-X responds equally well to picoMolar concentrations of 17α-estradiol and 17β-estradiol (FIGS. 3a and 3b). Finally, ER-X simply cannot represent expression of a protein derived from the targeted gene disruption used to generate ERKO mice, since ER-X is present at comparable levels in P7 wild-type and ERKO neocortex (FIG. 1b). Earlier studies of cellular variations in ER mRNA translation (Toran-Allerand, 1992) have provided data consistent with the hypothesis that some of the ER-α-like mRNA detected by in situ hybridization may actually represent ER-X mRNA. While estrogen binding and ER mRNA expression always co-localized, neurons expressing ER mRNA did not always exhibit nuclear binding, and there was no clear-cut relationship between the widespread hybridization signal (Miranda, 1992) and the limited extent of estrogen binding (Gerlach, 1983).

The SDS-PAGE conditions required to separate the ˜62-63 kDa protein are incompatible with retention of binding site integrity. Nevertheless, evidence suggests that the ˜62-63 kDa protein binds estradiol and, moreover, that this binding reaction may mediate activation of ERK1/2. The ˜62-63 kDa band and the estradiol binding site are both present in P7 ERKO neocortical membranes that contain neither ER-α nor ER-β. In ERKO mouse neocortex, 17α-estradiol and 17β-estradiol both activate ERK1/2: both also compete strongly for membrane binding of ³H-estradiol (FIG. 8). Levels of membrane binding are similar in ERKO and wild type neocortex, consistent with the observation that similar concentrations of the ˜62-63 kDa immunoreactive band are present in membranes from ERKO and wild-type P7 mice (FIG. 1). Finally, progesterone, which does not bind ER-α or ER-β but which does activate ERK in developing brain (Singh, 2001), is capable of competing with ³H-estradiol for the membrane binding site, albeit less effectively than 17α-estradiol and 17β-estradiol.

ER-X expression is developmentally regulated in both neocortex and uterus and is maximally expressed ˜P7-10. Wild-type and ERKO neocortical and uterine ER-x levels declined during the first postnatal month and became dramatically reduced in the adult, which expressed little ER-X (FIGS. 9a and 9b). Transient, neocortical expression of ER-X mimics the developmental pattern of estrogen binding (Gerlach, 1983). Since loss of functional ER-1 in ERKO mice did not appear to influence prenatal sexual development, it was concluded that development of the reproductive tract can occur in the absence of ER-mediated responsiveness (Lubahn, 1993). An alternate explanation is that early development may depend on another ER, such as ER-X.

Developmentally regulated estrogen receptors may be up-regulated and re-expressed in the adult brain. Previous studies have demonstrated that 17α-estradiol and 17β-estradiol protect against ischemic CNS injury, as well as neuronal cell death induced by exposure to peroxides or β-amyloid (reviewed in Green, 2000). The neuroprotective efficacy of 17α-estradiol has been interpreted as evidence of a direct antioxidant, as opposed to an ER-dependent mechanism (Behl, 1997; and Green, 1997). An alternative explanation is that responses to 17α-estradiol reflect activation of membrane ER-X response pathways. Developmentally regulated ERs, such as neocortical ER-α and ER-X, latent in the brain since development, may be re-expressed in the adult following injury due to ischemia, loss of trophic support or steroid deprivation. ER-X and its signaling pathways could therefore underlie not only the differentiative effects of estrogen in the developing brain but some of its neuroprotective actions in the adult (Green, 2000; Dubal, 1998; and Simpkins, 1997).

Data presented here demonstrate that the presence of a novel functional estrogen receptor associated with estradiol-induced activation of the MAPK cascade. Responses to estrogen during development and following injury are not necessarily mediated via either ER-α or ER-β, but could be mediated by ER-X. Association with CLMs positions ER-X uniquely to interact with co-localized signaling kinases, providing a novel mechanism for mediation of estrogen's influences on neuronal differentiation (Toran-Allerand, 1976), survival (Garcia-Segura, 2001), and plasticity (Matsumoto, 1981).

### REFERENCES

1. Simpkins et al. U.S. Pat. No. 5,554,601.
2. Simpkins et al. U.S. Pat. No. 5,843,934.
3. Anderson R. G. (1998) The caveolae membrane system. Annu. Rev. Biochem. 67:199-225.
4. Anuradha P., Khan S. M., Karthikeyan N., and Thampan R. V. (1994) The nonactivated estrogen receptor (naER) of the goat uterus is a tyrosine kinase. Arch. Biochem. Biophys. 309:195-204.
5. Arnold L. E., (1996) Sex differences in ADHD: conference summary. J. Abnorm. Child. Psychol. 24:555-569.
6. Aronica S. M., Kraus W. L., and Katzenellenbogen B. S. (1994) Estrogen action via the cAMP signaling pathway: stimulation of adenylate cyclase and cAMP-regulated gene transcription. Proc. Natl. Acad. Sci. USA 91:8517-8521.
7. Beato M., and Klug J. (2000) Steroid hormone receptors: an update. Hum. Reprod. Update 6:225-236.
8. Behl C., Skutella T., Lezoualc'h F., Post A., Widmann M., Newton J., and Holsboer F. (1997) Neuroprotection against oxidative stress by estrogens: structure-activity relationship. Mol. Pharmacol. 51:535-541.
9. Benten W. P., Stephan C., Lieberherr M., and Wunderlich F. (2001) Estradiol signaling via sequestrable surface receptors. Endocrinology 142(4):1669-1677.
10. Bickel P. E., Scherer P. E., Schnitzer J. E., Oh P., Lisanti M. P. and Lodish H. F. (1997) Flotillin and epidermal surface antigen define a new family of caveolae-associated integral membrane proteins. J. Biol. Chem. 272:13793-13802.
11. Blaustein J. D. (1992) Cytoplasmic estrogen receptors in rat brain: immunocytochemical evidence using three antibodies with distinct epitopes. Endocrinology 131:1336-1342.
12. Chiaia N., Foy M., and Teyler T. J. (1983) A simple method for fashioning small diameter concentric bipolar electrodes for stimulation of nervous tissues. Behav. Neurosci. 97:839-843.
13. Clark A. S., and Goldman-Rakic P. (1989) Gonadal hormones influence the emergence of cortical function in nonhuman primates. Behav. Neurosci. 103:1287-1295.
14. Das S. K., Taylor J. A., Korach K. S., Paria B. C., Dey S. K., and Lubahn D. B. (1997) Estrogenic responses in estrogen receptor-alpha deficient mice reveal a distinct estrogen signaling pathway. Proc. Natl. Acad. Sci. U.S.A 94:12786-12791.
15. Dubal D. B., Kashon M. L., Pettigrew L. C., Ren J. M., Finklestein S. P., Rau S. W., and Wise P. M. (1998) Estradiol protects against ischemic injury. J. Cereb. Blood Flow Metab. 18:1253-1258.
16. Dubal D. B., Zhu H., Yu J., Rau S. W., Shughrue P. J., Merchenthaler I., Kindy M. S., and Wise P. M. (2001) Estrogen receptor alpha, not beta, is a critical link in estradiol-mediated protection against brain injury. Proc. Natl. Acad. Sci. U.S.A 98:19552-19577.
17. Filardo E. J., Quinn J. A., Bland K. I. and Frackelton A. R. Jr. (2000) Estrogen-induced activation of Erk-1 and Erk-2 requires the G protein-coupled receptor homologue, GPR30, and occurs via trans-activation of the epidermal growth factor receptor through release of HB-EGF. Mol. Endocrinol. 14:1649-1660.
18. Fitzpatrick S. L., Funkhouser J. M., Sindoni D. M., Stevis P. E., Deecher D. C., Bapat A. R., Merchenthaler I., and Frail D. E. (1999) Expression of estrogen receptor-beta protein in rodent ovary. Endocrinology 140:2581-2591.
19. Friend K. E., Resnick E. M., Ang L. W., and Shupnik M. A. (1997) Specific modulation of estrogen receptor mRNA isoforms in rat pituitary throughout the estrous cycle and in response to steroid hormones. Mol. Cell Endocrinol. 131:147-155.
20. Garcia-Segura L. M., Olmos G. Tranque P. and Naftolin F. (1987) Rapid effects of gonadal steroids upon hypothalamic neuronal membrane ultrastructure. J. Steroid Biochem. 27:615-623.
21. Garcia-Segura L. M., Azcoitia I., and DonCarlos L. L. (2001) Neuroprotection by estradiol. Prog. Neurobiol. 63:29-60.
22. Gerlach J., McEwen B. S., Toran-Allerand C. D. and Friedman W. J. (1983) Perinatal development of estrogen receptors in mouse brain assessed by radioautography, nuclear isolation and receptor assay. Develop. Brain Res. 11:7-18.
23. Geschwind N., and Behan P. O. (1982) Left-handedness: association with immune disease, migraine and developmental learning disorders. Proc. Natl. Acad. Sci. USA 7:5097-5100.
24. Goldman P. S., Crawford H. T., Stokes L. P., Galkin T. W., and Rosvold H. E. (1974) Sex-dependent behavioral effects of cerebral cortical lesions in developing rhesus monkey. Science 186:540-542.
25. Green P. S., Bishop J., and Simpkins J. W. (1997) 17alpha-estradiol exerts neuroprotective effects on SK—N—SH cells. J. Neurosci. 17:511-515.
26. Green P. S., and Simpkins J. W. (2000) Neuroprotective effects of estrogens: potential mechanisms of action. Int. J. Dev. Neurosci. 18:347-358.
27. Gu Q., Korach K. S., and Moss R. L. (1999) Rapid action of 17beta-estradiol on kainate-induced currents in hippocampal neurons lacking intracellular estrogen receptors. Endocrinology 140:660-666.
28. Guo Z., Krucken J., Benten W. P., Wunderlich F. (2002) Estradiol-induced nongenomic calcium signaling regulates genotropic signaling in macrophages. J. Biol. Chem. 277:7044-7050.
29. Gutkind J. S. (2000) Regulation of mitogen-activated protein kinase signaling networks by G protein-coupled receptors. Sci. STKE 40:RE1. Review.
30. Hajek R. A., Robertson A. D., Johnston D. A., Van N. T., Tcholakian R. K., Wagner L. A., Conti C. J., Meistrich M. L., Contreras N., Edwards C. L., and Jones L. A. (1997) During development, 17alpha-estradiol is a potent estrogen and carcinogen. Environ. Health Perspect. 105 Suppl 3:577-581.
31. Hawkins M. B., Thornton J. W., Crews D., Skipper J. K., Dotte A., and Thomas P. (2000) Identification of a third distinct estrogen receptor and reclassification of estrogen receptors in teleosts. Proc. Natl. Acad. Sci. USA 97:10751-10756.
32. Huang C. S., Zhou J., Feng A. K., Lynch C., Klumperman J., DeArmond S. J., and Mobley W. C. (1999) Nerve growth factor signaling in caveolae-like domains at the plasma membrane. J. Biol. Chem. 274:36707-36714.
33. Huang J., Choudhri T. F., Winfree C. J., McTaggart R. A., Kiss S., Mocco J., Kim L. J., Protopsaltis T. S., Zhang Y., Pinsky D. J., and Connolly E. S. Jr. (2000) Postischemic cerebrovascular E-selectin expression mediates tissue injury in murine stroke. Stroke 31:3047-3053.
34. Iwabuchi K., Handa K., and Hakomori S. (2000) Separation of glycosphingolipid-enriched microdomains from caveolar membrane characterized by presence of caveolin. Methods Enzymol. 312:488-494.
35. Jones E. G. (1995) Cortical development and neuropathology in schizophrenia. Ciba. Found. Symp. 193:277-295.
36. Karthikeyan N., and Thampan R. V. (1996) Plasma membrane is the primary site of localization of the nonactivated estrogen receptor in the goat uterus: hormone binding causes receptor internalization. Arch. Biochem. Biophys. 325:47-57.
37. Kelly M. J., Moss R. L., and Dudley C. A. (1978) The effect of ovariectomy on the responsiveness of preoptic-septal neurons to microelectrophoresed estrogen. Neuroendocrin. 25:204-211.
38. Kelly M. J., and Wagner E. J. (1999) Estrogen Modulation of G-protein-coupled Receptors. Trends Endocrinol. Metab. 10:369-374.
39. Kuiper G. G., Enmark E., Pelto-Huikko M., Nilsson S., and Gustafsson J. A. (1996) Cloning of a novel receptor expressed in rat prostate and ovary. Proc. Natl. Acad. Sci. U.S.A 93:5925-5930.
40. Kuiper G. G., Carlsson B., Grandien K., Enmark E., Haggblad J., Nilsson S., and Gustafsson J. A. (1997) Comparison of the ligand binding specificity and transcript tissue distribution of estrogen receptors alpha and beta. Endocrinology 138:863-870.
41. Landers J. P., and Spelsberg T. C. (1992) New concepts in steroid hormone action: Transcription factors, proto-oncogenes and the cascade model for steroid regulation of gene expression. Crit. Rev. Eukaryotic Gene Expression 2:19-63.
42. Levin E. R. (2002) Cellular functions of plasma membrane estrogen receptors. Steroids. 67:471-475.
43. Liu P., Ying Y. S. and Anderson R. G. W. (1997) Platelet-derived growth factor activates mitogen-activated protein kinase in isolated caveolae. Proc. Natl. Acad. Sci. USA 94: 13666-13670.
44. Lubahn D. B., Moyer J. S., Golding T. S., Couse J. F., Korach K. S., and Smithies O. (1993) Alteration of reproductive function but not prenatal sexual development after insertional disruption of the mouse estrogen receptor gene. Proc. Natl. Acad. Sci. U.S.A 90:1162-1166.
45. MacLusky N. J., Naftolin F., Goldman-Rakic P. S. (1986) Estrogen formation and binding in the cerebral cortex and diencephalon of the newborn rhesus monkey. Proc. Natl. Acad. Sci. USA 83:513-516.
46. Marino M., Distefano E., Trentalance A., and Smith C. L. (2001) Estradiol-induced IP(3) mediates the estrogen receptor activity expressed in human cells. Mol. Cell. Endocrinol. 182:19-26.
47. Marshall C. J. (1995) Specificity of receptor tyrosine kinase signaling: transient versus sustained extracellular signal-regulated kinase activation. Cell. 80:179-185.
48. Matsumoto A., and Arai Y. (1981) Neuronal plasticity in the deafferented hypothalamic arcuate nucleus of adult female rats and its enhancement by treatment with estrogen. J. Comp. Neurol. 197:197-205.
49. McGookey D. J., Fagerberg K., and Anderson R. G. (1983) Filipin-cholesterol complexes form in uncoated vesicle membrane derived from coated vesicles during receptor-mediated endocytosis of low density lipoprotein. J. Cell Biol. 96:1273-1278.
50. Migliaccio A., Pagano M., and Aurricchio F. (1993) Immediate and transient stimulation of protein tyrosine phosphorylation by estradiol in MCF-7 cells. Oncogene 8:2183-2191.
51. Milner T. A., McEwen B. S., Hayashi S., Li C. J., Reagan L. P. and Alves S. E. (2001) Ultrastructural evidence that hippocampal alpha estrogen receptors are located at extranuclear sites. J. Comp. Neurol. 429:355-371.
52. Miranda R. C., and Toran-Allerand C. D. (1992) Developmental expression of estrogen receptor mRNA in the rat cerebral cortex: A non-isotopic in situ hybridization histochemistry study. Cerebral Cortex 2:1-15.
53. Nadal A., Ropero A. B., Laribi O., Maillet M., Fuentes E., and Soria B. (2000) Nongenomic actions of estrogens and xenoestrogens by binding at a plasma membrane receptor unrelated to estrogen receptor alpha and estrogen receptor beta. Proc. Natl. Acad. Sci. U.S.A 97:11603-11608.
54. Nethrapalli I. S., Singh M., Guan X., Guo Q. F., Lubahn D. B., Korach K. S. and Toran-Allerand C. D. (2001) Estrogen Elicits Src Phosphorylation in Mouse Neocortical Explants: An Upstream Event in Estrogen Activation of the MAP Kinase Cascade? Endocrinology 142:5145-5148.
55. Okamoto T., Schlegel A., Scherer P. E., and Lisanti M. P. (1998) Caveolins, a family of scaffolding proteins for organizing “pre-assembled signaling complexes” at the plasma membrane. J. Biol. Chem. 273:5419-5422.
56. Pennington B. F., Heaton R. K., Karzmark P., Pendleton M. G., Lehman R., and Shucard D. W. (1985) The neuropsychological phenotype in Turner syndrome. Cortex 21:391-404.
57. Petersen D. N., Tkalcevic G. T., Koza-Taylor P. H., Turi T. G., and Brown T. A. (1998) Identification of estrogen receptor beta2, a functional variant of estrogen receptor beta expressed in normal rat tissues. Endocrinology 139:1082-1109.
58. Pietras R. J., and Szego C. M. (1977) Specific binding sites for oestrogen at the outer surfaces of isolated endometrial cells. Nature 265:69-72.
59. Razandi M., Pedram A., Greene G. L., and Levin E. R. (1999) Cell membrane and nuclear estrogen receptors (ERs) originate from a single transcript: studies of ER-α and ER-β expressed in Chinese hamster ovary cells. Mol. Endocrinol. 13:307-319.
60. Razandi M., Oh P., Pedram A., Schnitzer J., and Levin E. R. (2002) ERs associate with and regulate the production of caveolin: implications for signaling and cellular actions. Mol. Endocrinol. 16:100-115.
61. Ros-Baro A., Lopez-Iglesias C., Peiro S., Bellido D., Palacin M., Zorzano A., and Camps M. (2001) Lipid rafts are required for GLUT4 internalization in adipose cells. Proc. Natl. Acad. Sci. USA 98:12050-12055.
62. Rothberg K. G., Ying Y. S., Kamen B. A., and Anderson R. G. (1990) Cholesterol controls the clustering of the glycophospholipid-anchored membrane receptor for 5-methyltetrahydrofolate. J. Cell Biol. 111:2931-2938.
63. Schlegel A., Volonte D., Engelman J. A., Galbiati F., Mehta P., Zhang X. L., Scherer P. E., and Lisanti M. P. (1998) “Crowded little caves”: structure and function of caveolae. Cell Signal. 10:457-463.
64. Schreihofer D. A., Resnick E. M., Soh A. Y., and Shupnik M. A. (1999) Transcriptional regulation by a naturally occurring truncated rat estrogen receptor (ER), truncated ER product-1 (TERP-1). Mol. Endocrinol. 13:320-329.
65. Schupf N., and Sergievsky G. H. (2002) Genetic and host factors for dementia in Down's syndrome. Br. J. Psychiatry. 180:405-410.
66. SétálóG. Jr., Singh M., Guan X., and Toran-Allerand C. D. (2001) Cellular localization of estradiol-induced phospho-ERK1/2 in mouse cerebral cortical explants: the roles of heat shock protein 90 and MEK2. J. Neurobiol. 50:1-12.
67. SétálóG. Jr., Singh M., Guan X., and Toran-Allerand C. D. (2002) Cellular localization of estradiol-induced phospho-ERK1/2 in mouse cerebral cortical explants: the roles of heat shock protein 90 and MEK2. J. Neurobiol. 50:1-12.
68. Shughrue P. J., Stumpf W. E., MacLusky N. J., Zielinski J. E., and Hochberg R. B. (1990) Developmental changes in estrogen receptors in mouse cerebral cortex between birth and postweaning: studied by autoradiography with 11 beta-methoxy-16 alpha-[¹²⁵I] iodoestradiol. Endocrinology 126:1112-1124.
69. Shughrue P. J., Lane M. V., and Merchenthaler I. (1997) Comparative distribution of estrogen receptor-alpha and -beta mRNA in the rat central nervous system. J. Comp. Neurol. 388:507-525.
70. Shughrue P. J., Askew G. R., Dellovade T. L., and Merchenthaler I. (2002) Estrogen-binding sites and their functional capacity in estrogen receptor double knockout mouse brain. Endocrinology 143:1643-1650.
71. Simpkins J. W., Rajakumar G., Zhang Y. Q., Simpkins C. E., Greenwald D., Yu C. J., Bodor N., and Day A. L. (1997) Estrogens may reduce mortality and ischemic damage caused by middle cerebral artery occlusion in the female rat. J. Neurosurg. 87:724-730.
72. Singer C. A., Figueroa-Masot X. A., Batchelor R. H., and Dorsa D. M. (1999) The mitogen-activated protein kinase pathway mediates estrogen neuroprotection after glutamate toxicity in primary cortical neurons. J. Neurosci. 19:2455-2463.
73. Singh M., SétálóJr. G., Guan X., Warren M., and Toran-Allerand C. D. (1999) Estrogen-induced activation of MAP Kinase (ERK) in cerebral cortical explants: Convergence of estrogen and neurotrophin signaling pathways. J. Neurosci. 19:1179-1188.
74. Singh M., SétálóJr. G., Guan X., Frail D. F., and Toran-Allerand C. D. (2000) Estrogen-induced activation of the MAP kinase cascade in the cerebral cortex of estrogen receptor-α knock-out mice. J. Neurosci. 20:1694-1700.
75. Singh M. (2001) Ovarian hormones elicit phosphorylation of Akt and extracellular-signal regulated kinase in explants of the cerebral cortex. Endocrine 14:407-415.
76. Smart E. J., Ying Y. S., Mineo C., and Anderson R. G. (1995) A detergent-free method for purifying caveolae membrane from tissue culture cells. Proc. Natl. Acad. Sci. U.S.A 92:10104-10108.
77. Stauffer S. R., Coletta C. J., Tedesco R., Nishiguchi G., Carlson K., Sun J., Katzenellenbogen B. S., and Katzenellenbogen J. A. (2000) Pyrazole ligands: Structure-affinity/activity relationships and estrogen receptor-α selective agonists. J. Med. Chem. 43:4934-4947.
78. Strauss E., Wada J., and Hunter M. (1992) Sex-related differences in the cognitive consequences of early left hemisphere lesions. J. Clin. Exp. Neuropsychol. 14:738-748.
79. Subtil A., Gaidarov I., Kobylarz K., Lampson M. A., Keen J. H., and McGraw T. E. (1999) Acute cholesterol depletion inhibits clathrin-coated pit budding. Proc. Natl. Acad. Sci. USA 96:6775-6780.
80. Sukovich D. A., Mukherjee R., and Benfield P. A. (1994) A novel, cell-type-specific mechanism for estrogen receptor-mediated gene activation in the absence of an estrogen-responsive element. Mol. Cell. Biol. 14:7134-7143.
81. Tallal P. (1991a) Hormonal influences in developmental learning disabilities. Psychoneuroendocrinology 16:203-212.
82. Tallal P., and McEwen B. (1991b) Neuroendocrine effects on brain development and cognition. Psychoneuroendocrinology 16:3-6.
83. Tang M. X., Jacobs D., Stern Y., Marder K., Schofield P., Gurland, Andrews H., and Mayeux R. (1996) Effect of oestrogen during menopause on risk and age of onset of Alzheimer's disease. Lancet 348:429-432.
84. Thuresson-Klein A., Moawad A. H., and Hedqvist P. (1985) Estrogen stimulates formation of lamellar bodies and release of surfactant in the rat fetal lung. Am. J. Obstet. Gynecol. 151:506-514.
85. Toran-Allerand C. D. (1976) Sex steroids and the development of the newborn mouse hypothalamus and preoptic area in vitro: Implications for sexual differentiation. Brain Res. 106:407-412.
86. Toran-Allerand C. D. (1980) Sex steroids and the development of the newborn mouse hypothalamus and preoptic area in vitro: II. Morphological correlates and hormonal specificity. Brain Res. 189:413-427.
87. Toran-Allerand C. D., Miranda R. C., Hochberg R. B., and MacLusky N. J. (1992) Cellular variations in estrogen receptor mRNA translation in the developing brain: Evidence from combined ¹²⁵I-estrogen autoradiography and non-isotopic in situ hybridization histochemistry. Brain Res. 576:25-41.
88. Toran-Allerand C. D. (1996) The estrogen/neurotrophin connection during neural development: is co-localization of estrogen receptors with the neurotrophins and their receptors biologically relevant? Dev. Neurosci. 18:36-48.
89. Toran-Allerand C. D., Singh M., and SétálóJr. G. (1999) Estrogen action in the brain: New players in an old story. Frontiers in Neuroendocrinology 20:97-121.
90. Toran-Allerand C. D. (2000) Novel sites and mechanisms of estrogen action in the brain. In: Neuronal and Cognitive Effects of Oestrogens, Wiley, Chichester (Novartis Foundation Symposium 230), pages 56-73.
91. Traverse S., Gomez N., Paterson H., Marshall C., and Cohen P. (1992) Sustained activation of the mitogen-activated protein (MAP) kinase cascade may be required for differentiation of PC12 cells. Comparison of the effects of nerve growth factor and epidermal growth factor. Biochem. J. 288:351-355.
92. Tremblay G. B., Tremblay A., Copeland N. G., Gilbert D. J., Jenkins N. A., Labrie F., and. Giguere V. (1997) Cloning, chromosomal localization, and functional analysis of the murine estrogen receptor beta. Mol. Endocrinol. 11:353-365.
93. Trotter A., and Pohlandt F. (2000) The replacement of oestradiol and progesterone in very premature infants. Ann. Med. 32:608-614.
94. Tsuchiya K., Ikeda K., Niizato K., Watabiki S., Anno M., Taki K., Haga C., Iritani S., and Matsushita M. (2002) Parkinson's disease mimicking senile dementia of the Alzheimer type: a clinicopathological study of four autopsy cases. Neuropathology 22:77-84.
95. Wade C. B., Robinson S., Shapiro R. A., and Dorsa D. M. (2001) Estrogen receptor (ER) alpha and ERbeta exhibit unique pharmacologic properties when coupled to activation of the mitogen-activated protein kinase pathway. Endocrinology 142:2336-2342.
96. Watson C. S., Norfleet A. M., Pappas T. C., and Gametchu B. (1999) Rapid actions of estrogens in GH3/B6 pituitary tumor cells via a plasma membrane version of estrogen receptor-alpha. Steroids 64:5-13.
97. Watson F. L., Heerssen H. M., Bhattacharyya A., Klesse L., Lin M. Z., and Segal R. A. (2001) Neurotrophins use the Erk5 pathway to mediate a retrograde survival response. Nat Neurosci. 4:981-988.
98. Watters J. J., Campbell J. S., Cunningham M. J., Krebs E. G., and Dorsa D. M. (1997) Rapid membrane effects of steroids in neuroblastoma cells: effects of estrogen on mitogen activated protein kinase signalling cascade and c-fos immediate early gene transcription. Endocrinology 138:4030-4033.
99. White R., Lees J. A., Needham M., Ham J., and Parker M. (1987) Structural organization and expression of the mouse estrogen receptor. Mol. Endocrinol. 1:735-744.
100. Wyckoff M. H., Chambliss K. L., Mineo C., Yuhanna I. S., Mendelsohn M. E., Mumby S. M., and Shaul P. W. (2001) Plasma membrane estrogen receptors are coupled to endothelial nitric-oxide synthase through Galpha(i). J. Biol. Chem. 276:27071-27076.

1. An isolated mammalian cell-surface estrogen receptor characterized by
(a) a non-stereospecific binding affinity for 17α-estradiol and 17β-estradiol;
(b) at least one epitope in common with the ligand-binding domain of ER-α; and
(c) increased presence at caveolar or caveolar-like microdomains of cells on which the receptor is present.

2. (canceled)

3. A composition of matter comprising a lipid membrane, other than that of an intact cell, comprising the receptor of claim 1 operably situated therein.

4. (canceled)

5. A method for determining whether an agent specifically binds to the receptor of claim 1 which comprises
(a) contacting the receptor with the agent under suitable conditions;
(b) detecting the presence of any complex formed between the receptor and the agent; and
(c) determining whether the complex detected in step (b) is the result of specific binding between the agent and receptor, thereby determining whether the agent specifically binds to the receptor.

6. (canceled)

7. (canceled)

8. A method for determining the affinity with which an agent binds to the receptor of claim 1 relative to that with which a known ligand binds the receptor, which comprises
(a) concurrently contacting the receptor with both the agent and a ligand that binds the receptor with a known affinity under conditions which permit the formation of a complex between the receptor and the ligand;
(b) determining the amount of complex formed between the agent and the receptor; and
(c) comparing the amount of complex determined in step (b) with the amount of complex formed between the agent and the receptor in the absence of the ligand, wherein (i) a ratio of agent in the complex determined in step (c) to that determined in step (b) greater than 2 indicates that the agent binds to the receptor with less affinity than does the ligand, (ii) a ratio of less than 2 indicates that the agent binds to the receptor with greater affinity than does the ligand, and (iii) a ratio of 2 indicates that the agent and ligand bind to the receptor with the same affinity.

9. (canceled)

10. A method for determining whether an agent is an agonist of the receptor of claim 1, which comprises
(a) contacting the receptor with the agent under conditions which permit (i) the formation of a complex between the receptor and a known agonist of the receptor, and (ii) the generation of a detectable signal upon formation of a complex between the receptor and the known agonist; and
(b) determining whether a detectable signal is generated in step (a), the generation of such signal indicating that the agent is an agonist of the receptor.

11. (canceled)

12. A method for determining whether an agent is an antagonist of the receptor of claim 1, which comprises
(a) contacting the receptor with the agent, in the presence of a known agonist, under conditions which permit (i) the formation of a complex between the receptor and the agonist, and (ii) the generation of a detectable signal upon formation of a complex between the receptor and the agonist; and
(b) comparing the signal, if any, generated in step (a) with the signal generated in the absence of the agent, the generation of a signal in the agent's absence greater than that generated in the agent's presence indicating that the agent is an antagonist.

13. (canceled)

14. A method for activating the MAP kinase pathway of a cell having on its surface the receptor of claim 1 comprising contacting the cell with a concentration of 17α-estradiol of at least 0.1 pM and less than 100 pM under conditions permitting the 17α-estradiol to bind to the receptor, thereby activating the MAP kinase pathway in the cell.

15. (canceled)

16. (canceled)

17. A method for treating a subject afflicted with a neurodegenerative disorder, comprising administering to the subject an amount of 17α-estradiol sufficient to raise the subject's plasma 17α-estradiol concentration to at least 0.1 pM and less than 100 pM, thereby treating the subject.

18. A method for delaying the onset of a neurodegenerative disorder in a subject, comprising administering to the subject an amount of 17α-estradiol sufficient to raise the subject's plasma 17α-estradiol concentration to at least 0.1 pM and less than 100 pM, thereby delaying the onset of the neurodegenerative disorder in the subject.

19. (canceled)

20. (canceled)

21. (canceled)

22. (canceled)

23. A method for treating a subject afflicted with a neurodevelopmental disorder, comprising administering to the subject an amount of 17α-estradiol sufficient to raise the subject's plasma 17α-estradiol concentration to at least 0.1 pM and less than 100 pM, thereby treating the subject.

24. (canceled)

25. (canceled)

26. (canceled)

27. (canceled)

28. A method for treating a subject afflicted with a sexually dimorphic childhood disorder of cognition, comprising administering to the subject an amount of 17α-estradiol sufficient to raise the subject's plasma 17α-estradiol concentration to at least 0.1 pM and less than 100 pM, thereby treating the subject.

29. (canceled)

30. (canceled)

31. (canceled)

32. (canceled)

33. (canceled)

34. A method for treating a subject afflicted with a uterine disorder, comprising administering to the subject an amount of 17α-estradiol sufficient to raise the subject's plasma 17α-estradiol concentration to at least 0.1 pM and less than 100 pM, thereby treating the uterine disorder in the subject.

35. (canceled)

36. (canceled)

37. A method for treating a subject afflicted with a pulmonary disorder, comprising administering to the subject an amount of 17α-estradiol sufficient to raise the subject's plasma 17α-estradiol concentration to at least 0.1 pM and less than 100 pM, thereby treating the subject.

38. (canceled)

39. (canceled)

40. A composition comprising (a) a pharmaceutically acceptable carrier and (b) a dose of 17α-estradiol which, when administered to a subject, is sufficient to raise the subject's plasma 17α-estradiol concentration to at least 0.1 pM and less than 100 pM.

41. An article of manufacture comprising (a) a packaging material having therein an amount of 17α-estradiol sufficient, upon administration to a subject, to raise the subject's plasma 17α-estradiol concentration to at least 0.1 pM and less than 100 pM, and (b) a label indicating a use of the 17α-estradiol for treating a disorder selected from the group consisting of a neurodegenerative disorder, a neurodevelopmental disorder, a sexually dimorphic childhood disorder of cognition, a uterine disorder, and a pulmonary disorder.


SEQUENCE LISTING (3 sequences)

SEQ ID NO: 1 - DNA, 23 bp, Artificial Sequence
  Probe/Primer
cggtctacggccagtcgggcatc

SEQ ID NO: 2 - DNA, 24 bp, Artificial Sequence
  Probe/Primer
gtagaaggcgggagggccggtgtc

SEQ ID NO: 3 - DNA, 24 bp, Artificial Sequence
  Probe/Primer
gctgaccgcttcctcgtgctttac